(19) United States
(12) Patent Application Publication
(10) Pub. No.: US 2025/0255596 A1
(43) Pub. Date: Aug. 14, 2025

(54) CATHETER TREATMENT SEGMENT

(71) Applicant: Sleep Solutions of Texas, LLC, Tyler, TX (US)

(72) Inventor: Raghavendra Vitthalrao GHUGE, Tyler, TX (US)

(73) Assignee: Sleep Solutions of Texas, LLC, Tyler, TX (US)

(21) Appl. No.: 19/051,691

(22) Filed: Feb. 12, 2025

Related U.S. Application Data
(60) Provisional application No. 63/552,273, filed on Feb. 12, 2024.

(51) Int. Cl. A61B 17/02; A61B 17/00; A61B 18/00; A61B 18/24; A61M 29/02; A61N 5/06

(52) U.S. Cl. CPC A61B 17/0218; A61B 18/24; A61M 29/02; A61N 5/0601; A61N 5/062; A61B 2017/00044; A61B 2017/00061; A61B 2017/00106; A61B 2017/00893; A61B 2017/0237; A61B 2018/00369; A61M 2202/07; A61M 2202/09; A61M 2205/3313; A61M 2205/3375; A61M 2210/125; A61M 2230/06; A61N 2005/0602

(57) ABSTRACT

Treatment segments of a catheter for heart valve treatment have a distal catheter body having a collapsible and expandable through conduit operatively connected to a collapsible and expandable valve cusp enclosure. The through conduit has a first end sealed by a first annular valve and a second end sealed by a second annular valve, which each have an elastic body, a distal end connected to the valve cusp enclosure, and a proximal end sealingly engaged to the distal catheter body in a deployed, closed position. The through conduit and the distal ends of each of the first and second annular valves are configured to expand with the valve cusp enclosure at a rate proportionate to a patient's blood pressure and a volume of blood displaced during expansion, and the elastic body and proximal ends thereof are configured to open and close in response to systolic and diastolic blood flow.

### TECHNICAL FIELD

This application relates to catheters, more particularly, to catheters having a treatment segment for the aortic valve that has a valve cusp enclosure to create a bloodless field around at least one aortic valve cusp while providing a dual valved through conduit for blood flow from the ventricle to the aorta.

### BACKGROUND

Catheters are used in various procedures for delivering therapeutic means to a treated site (e.g., body organ or passageway such as blood vessels). In many cases, a small inflatable balloon is guided to the treatment site. Once the balloon is in place, it is inflated by the operator to affix it in place, to expand a blocked vessel, to place treatment means (e.g., a stent) and/or to deliver surgical tools (e.g., knives, drills, etc.) to a desired site. One such procedure is transcatheter aortic valve replacement (TAVR), which is a minimally invasive method. This is typically used to treat aortic stenosis instead of open-heart surgery. Here, the aortic valve cusps are hyperextended radially outward and an artificial valve is positioned therebetween. The risks of the procedure include post-placement anticoagulation, blood vessel complications, valve slippage or leaking, stroke, heart arrhythmias, myocardial infarction, infection, and sometimes death.

U.S. Pat. No. 7,744,620 discloses a valvuloplasty catheter that uses one or more balloons to hyperextend a cusp of the aortic valve, thereby dislodging calcium deposits therefrom. In the embodiment of FIG. 8 thereof, a perfusion channel is present inside the balloon catheter and a single one-way valve that passively opens to allow blood flow. The perfusion channel closes when returning blood flow occurs. The blood is ventricular blood flowing into aorta. This is a universal need of all catheters that operate on the aortic valve and require blood flow from ventricle to exit into the aorta. The problem with this perfusion channel is that there is no method or design to address preferential flow, regulated volume or pressure of blood flow and thus greater risk of brain injury and organ damage due to lack of adequate perfusion pressure or blood flow. Moreover, the single one-way valve allows some antegrade flow of blood to reduce the resistance and prevent distal migration of the catheter. But a single valve is too weak to prevent retrograde flow of blood at high pressure. The pressure distal to the valve in the ventricle drops considerably and leaves the catheter in a position where it will collapse if it is flexible enough to be advanced through the arterial system of the subject. If the catheter is too rigid to withstand the variations of blood pressure below this valve, then it will not advance through the arterial tree of the subject.

There is a need for improved procedures and catheters that reduce the risks noted above, especially a catheter that is collapsible and flexible such that it can travel through the arterial system, and that has a means for continued blood flow of the patient without the need for a cardiopulmonary bypass pump. Moreover, there is a need to remodel and/or restore the aortic valve rather than require aortic valvuloplasty or aortic valve replacement after severe aortic stenosis with advanced calcification and fibrosis.

### SUMMARY

In one aspect, treatment segments of a catheter for heart valve treatment have a distal catheter body having a collapsible and expandable through conduit operatively connected to a collapsible and expandable valve cusp enclosure. The through conduit has a first end sealed by a first annular valve and a second end sealed by a second annular valve, which each have an elastic body, a distal end connected to the valve cusp enclosure, and a proximal end sealingly engaged to the distal catheter body in a deployed, closed position. The through conduit and the distal ends of each of the first and second annular valves are configured to expand with the valve cusp enclosure at a rate proportionate to a patient's blood pressure and a volume of blood displaced during expansion, and the elastic body and proximal ends thereof are configured to open and close in response to systolic and diastolic blood flow.

In all aspects, the elongate body of the through conduit is configured to retain blood between systolic opening and diastolic closing of the first and second annular valves. The first annular valve and the second annular valve can be each frustoconically-shaped in a deployed, closed position.

In one aspect, the valve cusp enclosure has one or more ventricular cusp and aortic cusp pairs, wherein each pair has attached ends at the through conduit, the attached ends being spaced apart and having a catheter working port exit therebetween, each pair defines a pocket therebetween, and each pair has free ends configured to engage a patient's inferior surface of the aortic valve and superior surface of the aortic valve, respectively. The catheter working port exit is a terminal end of a port that extends radially from the distal catheter body through the through conduit. The port is expandable and collapsible between a deployed state and a transport state, respectively. In one embodiment, the pocket is configured to receive a single one of a patient's valve cusp therein. In another embodiment, the pocket is configured to receive two or more of a patient's valve cusps therein.

The one or more ventricular cusp and aortic cusp pairs each comprise an inflatable balloon having a plurality of individually controlled inflation chambers. The aortic cusp of the one or more ventricular cusp and aortic cusp pairs is controllably inflatable to maintain blood flow to the right and left coronary arteries. The through conduit has a length configured to position the first annular valve superior relative to the right and left coronary arteries. The aortic cusp is controllably inflatable to increase blood flow to the left and right coronary arteries.

The one or more ventricular cusp and aortic cusp pairs each comprise a plurality of telescoping, operatively expandable links housed inside a biomaterial. Each of the plurality of telescoping, operatively expandable links terminate with a foot configured to engage the respective inferior or superior surface of the annulus of the aortic valve. Each of the plurality of telescoping, operatively expandable links comprise one or more elbow joints defining an axis of rotation about which the more distal link is rotatable.

In another aspect, a treatment segment can include an electrocardiogram sensor, and the through conduit and the distal ends of each of the first and second annular valves are configured to expand controllably based on data from the electrocardiogram sensor.

In another aspect, methods of remodeling a cusp of a heart valve of a patient in need thereof are disclosed herein. The method includes introducing a catheter having a heart valve treatment segment as described herein to a target heart valve, deploying the collapsible and expandable valve cusp enclosure into an expanded state in which a cusp of a heart valve in need of remodeling is enclosed in an isolated pocket and simultaneously expanding the through conduit into a corresponding expanded state, removing blood from the isolated pocket via the catheter to form a bloodless field surrounding the cusp of the heart valve, remodeling the cusp of the heart valve, collapsing the valve cusp enclosure, and removing the catheter from the patient. Introducing the catheter can include feeding the catheter through a patient's artery based on robotics in a terminal cap guided by fiberoptic imaging or infrared or IVUS videography or EKG sensors that seek cardiac sinus node electric homing, or a combination thereof. Deploying the valve cusp enclosure can include inflating a plurality of balloon segments with a fluid. Determining a lipid burden or calcium burden or fibrous scar tissue can include application of near-infrared spectroscopy plus intravascular ultrasound or a capacitive micromachines ultrasound transducer and fiberoptic cameras.

In one embodiment, the method includes advancing a tool through the catheter into the bloodless field and remodeling by one or more of:
i) removing fibrotic, calcific, and/or lipid laden material from a surface of the cusp using the tool;
ii) smoothing a surface of the cusp using the tool;
iii) preparing a surface of the cusp to accept a resurfacing material using the tool; and
iv) applying a resurfacing material to a surface of the cusp.

The “removing,” item (i) above, can include a laser treatment of the surface of the cusp. The resurfacing material can be an elastin and/or stem cells. In another embodiment, the resurfacing material can be a drug treatment, such as collagen and/or carbon dots. When the resurfacing material is collagen, the method includes activating the collagen by application of an activating wavelength of energy.

### BRIEF DESCRIPTION OF THE DRAWINGS

FIG. 1 is a side perspective view of one embodiment of a catheter in its collapsed form for travel through an artery of a patient and showing some of the interior structures thereof.

FIG. 2 is a longitudinal cross-section of the first embodiment in a deployed state showing some of the interior structures thereof.

FIG. 3 is a longitudinal cross-section of the first embodiment in a deployed state in the aortic root with one cusp of an aortic valve in a bloodless field defined by lobes of the catheter.

FIG. 4 is a transverse cross-sectional view taken along line 4-4 in FIG. 3, but only showing the catheter and the aortic valve cusp present in the pocket.

FIG. 5 is a longitudinal cross-section of a second embodiment of a catheter in a deployed state.

FIG. 6 is a longitudinal cross-section of a third embodiment of a catheter in a deployed state.

FIG. 7 is a longitudinal cross-section of a fourth embodiment of a catheter in a partially deployed state.

FIG. 8 is a longitudinal cross-section of a fifth embodiment of a catheter in a fully deployed state.

FIG. 9 is a view of the distal end of the catheter of FIG. 8 from the ventricle of the heart as represented by the line 9-9.

FIG. 10 is a side perspective view of a terminal cap for any of the embodiments of catheters disclosed herein and showing some of the interior structures of the terminal cap.

FIG. 11 is a plan view of the proximal end of the terminal cap of FIG. 10.

FIG. 12 is an illustration of the cusps of the aortic valve.

FIG. 13 is a left ventricle pressure volume curve.

FIG. 14 is an aorta pressure volume curve.

FIG. 15 is a chart of aortic valve cusp dimensions.

FIG. 16 is a series of mathematical equations corresponding to the anatomy of the aortic valve.

FIG. 17 is a chart of values having the tangential shear calculated therein.

FIG. 18 is a chart of the velocity of blood and the smallest concentric area of the aortic valve relative to the degree of stenosis present in the aortic valve.

FIG. 19 is a chart of computer derived dimensions for remodeling the aortic valve.

FIG. 20 is a chart of computer-generated calculations and resulting incremental increase in thickness of the left coronary cusp of the aortic valve after remodeling.

FIG. 21 is a graph of the left coronary cusp height against remodel thickness in millimeters from the data in FIG. 20.

FIG. 22A is a series of mathematical equations related to blood flow and tangential sheer.

FIG. 22B presents graphs, (i) a cardiac pressure volume curve of the left ventricle and (ii) an aortic pressure volume curve thereof.

FIG. 23 is an anatomy illustration of the ventricle and aortic valve and corresponding mathematical equations.

### DETAILED DESCRIPTION

The following detailed description will illustrate the general principles of the invention, examples of which are additionally illustrated in the accompanying drawings. In the drawings, like reference numbers indicate identical or functionally similar elements.

The catheters disclosed herein enable treatment of aortic stenosis and/or ankylosis in its early stages, such as when mild or moderate, but is also suitable if it is severe. If treated early, it may be possible to prevent left ventricular longitudinal or concentric remodeling or hypertrophy and left ventricular fibrosis. Moreover, it may be possible to prevent right ventricular hypertension, left atrial enlargement, and decreased left ventricular systolic function. The catheters will enable a medical professional to remodel the aortic valve cusps to return flexibility and mobility thereto by any one or more of the following: (i) remove calcification, (ii) synthesize fibrosa epithelium on the aortic side of the cusp, (iii) synthesize ventricularis epithelium on the ventricular side of the cusp, (iv) synthesize spongiosa inside the body of the cusp, (v) remove lipid material from the sub-epithelium of the cusp, and (vi) remodel the cusps morphology to its more native valve shape.

The examples discussed herein are focused on the aortic valve. The heart has three other valves as well, the mitral valve, pulmonary valve, and tricuspid valve. The catheter and systems disclosed herein can be used to remodel cusps of the other valves too.

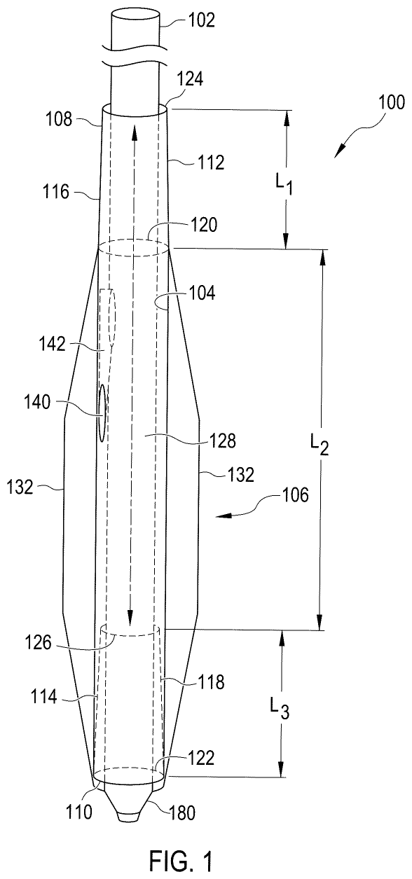

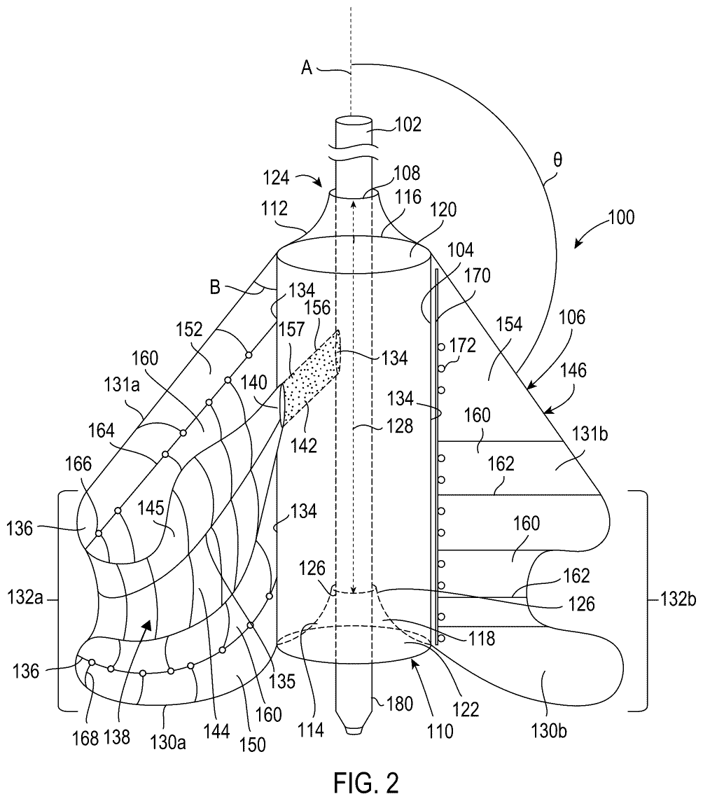

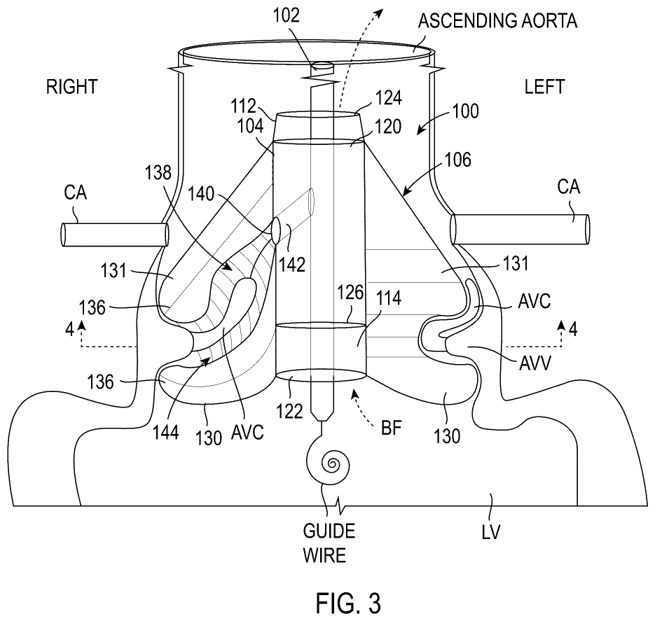

Turning to FIGS. 1-3, a first embodiment of a treatment segment of a catheter, generally referred to by the reference number 100, for heart valve treatments is shown in a transport state in FIG. 1 and in a deployed state in FIGS. 2 and 3. The treatment segment 100 has a distal catheter body 102 having a collapsible and expandable through conduit 104 in surrounding relationship to the distal catheter body 102 and operatively connected to a collapsible and expandable valve cusp enclosure 106. In the transport state, the components of the treatment segment are collapsed against the catheter body and have a profile small enough to be transported through a patient's arteries and aorto-ventricular system and flexible enough to navigate through the aorta, pulmonary artery, etc.

The diameter of the treatment segment 100 may range from 6 to 40 French, more preferably 12 French (12F) to 18 French (18F). The diameter can be customized to an individual's circulatory system size. The length of the treatment segment 100 has an overall length that can be customized to fit the height of the aortic root, location of the ostia of the right coronary artery and the left coronary artery origins, the height of the sinus of Valsalva and the structure of the left ventricle outflow tract of the intended patient. The width of the treatment segment 100 is customized to fit the ascending aortic diameter, aortic root diameter, aortic valve annular diameter, valvular opening (restricted by aortic stenosis) and the diameter of the ventricular outflow tract of the intended patient.

The through conduit 104 has a first end 108 sealed by a first annular valve 112 and a second end 110 sealed by a second annular valve 114, and each of the first and second annular valves 112, 114 have an elastic body 116, 118, a distal end 120, 122 connected to the valve cusp enclosure and a proximal end 124, 126, respectively, sealingly engaged to the distal catheter body in a deployed, valves closed position as shown in FIG. 2. The through conduit 104 and the distal ends 120, 122 of each of the first and second annular valves 112, 114 are configured to expand with the valve cusp enclosure 106 at a rate proportionate to a patient's blood pressure and a volume of blood displaced during expansion, and the elastic body 116, 118 and proximal ends 124, 126 of each of the first and second annular valves 112, 114 are configured to open and close in response to systolic and diastolic blood flow, respectively, thereby moving the proximal end 124, 126 of each in and out of sealing engagement with the distal catheter body 104.

In other words, each of the first and second annular valves 112, 114 are configured to allow blood to flow from the ventricle into the aorta. The second annular valve 114 has a proximal end 126 that is expansible (elastic) and inflates with each heartbeat from the pressure generated by the blood pressure as the ventricle generates a systolic force and then deflates due to its elastic nature during diastole and hugs the distal catheter body 104 such that there is no reverse flow. This allows blood to flow to the proximal end 124 of the first annular valve 112 during systole and not during diastole. The systolic blood pressure transmitted proximally due to opening of the proximal end 126 of the second annular valve 114 now allows the blood to flow through the first annular valve 112, distal to proximal, and the pressure expands the proximal end 124 of the first annular valve 112 with each stroke volume of blood delivered to it. The proximal end 124 is elastic and deflates during diastole; thus, hugging the distal catheter body 104 and preventing reversal of blood flow (BF), labeled in FIG. 3. In other words, blood can continue to flow through the heart when a procedure is being performed, thereby enabling a longer period of time for treatment procedures.

The through conduit 104 has an elongate body 128 configured to retain blood between systolic opening and diastolic closing of the first and second annular valves 112, 114. The first annular valve 112 and the second annular valve 114 are each frustoconically-shaped in a deployed, valves closed position as shown in FIG. 2 with the proximal ends 124, 126 closed onto the distal catheter body 102. The through conduit 104 has a length L configured to position the first annular valve 112 superior relative to the right and left coronary arteries. As seen in FIG. 1, the length L has been labeled as length portions L1 for the first annular valve 112, L2 for the portion of the through conduit between the proximal end of the second annular valve 114 and the distal end of the first annular valve 112, and L3 for the length of the second annular valve 114. L1 can range from 1 cm to 5 cm. L2 can range from 4 cm to 20 cm. L3 can range from 2 to 6 cm.

With reference to FIG. 3, in a deployed state at the left ventricle (LV) aortic valve annulus (AVV), the second end 110 of the treatment segment 100 including a distal-most lobe 130 of the valve cusp enclosure 106 and the second annular valve 114, which can be referred to as a distal ventricular valve. The treatment segment 100 is positioned with its second end 110 in the ventricular outflow tract below the aortic valve cusps (AVC) and in the deployed state the distal-most lobe 130 engages the inferior surface of the AVV. The first end 108 of the treatment segment 100 includes the first annular valve 112, which can be referred to as a proximal aortic valve. The treatment segment is positioned with its first end 108 in the aortic root cranially positioned relative to or superior to both the right coronary artery (right CA) and the left coronary artery (left CA).

Each of the first and second annular valves 112, 114 can be made of a solid flexible elastic polymer such as polytetrafluoroethylene or Dacron® by DuPont. The solid flexible elastic polymer may be thicker at the distal end and gradually becomes thinner proximally, such that the proximal end has a specific retractive property. In one embodiment, the proximal end has a stiffness of the elastic property that enables the valve to open at pressures of 150 mm of Hg to 200 mm of Hg. In another embodiment, the proximal end has a stiffness of the elastic property that enables the valve to open at pressures of 120 mm of Hg to 150 mm of Hg. In yet another embodiment, the proximal end has a stiffness of the elastic property that enables the valve to open at pressures of 90 mmm of Hg to 120 mm of Hg. The selected pressure range will vary from patient to patient based on historical blood pressure demonstrated prior to the procedure.

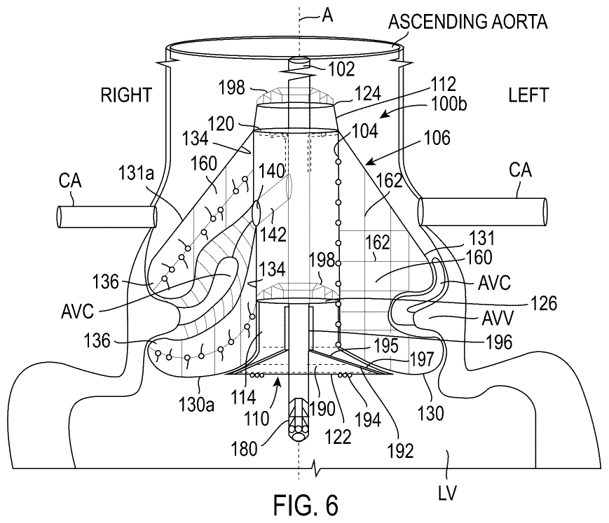

As noted above, each of the first and second annular valves 112, 114 have their respective distal ends 120, 122 configured to expand with the valve cusp enclosure 106 at a rate proportionate to a patient's blood pressure and a volume of blood displaced during expansion. This expansion can be accomplished by robotics (see FIGS. 6-9), a multi-segmented balloon (see FIGS. 1-3), or electrical signals (wired or wireless), e.g., activation of a shape memory alloy. The diameter achieved by any one of these expansion means is adjustable and can be configured to the individual size of the anatomy and hemodynamics of circulation and blood pressure or cardiac output of the intended patient.

Continuing to refer to FIGS. 1-3, in all embodiments, the valve cusp enclosure 106 has a first ventricular cusp and aortic cusp pair 132a that are attached to and extend from the through conduit 104. The attachment portion is referred to herein as respective attached ends 134 of each of the ventricular cusp and the aortic cusp of said pair 132a. The ventricular cusp 130 and the aortic cusp 131 are spaced apart proximate a working port exit 140 that is positioned therebetween and, when inflated/deployed, define a pocket 138 shaped to receive an aortic valve cusp (AVC) cusp therebetween. The pair 132a are permanently joined to one another by opposing vertical segments 135 to define the pocket 138. The vertical segments 135 extend radially from the attached ends 134 to the free end 136 of the cusps. In the embodiment of FIGS. 2-4 that treats one valve, the vertical segments 135 deploy simultaneously with the deployment of the two (aortic and ventricular) cusps. Each of the ventricular cusp 130 and the aortic cusp's 131 free end 136 is configured to engage a patient's inferior surface of the aortic valve and superior surface of the aortic valve, respectively, as shown in FIG. 3, which forms a fluid-tight seal to the AVV so that a bloodless field can be created around the AVC.

In one embodiment, the port 142 and its exit 140 are merely a part of an inflatable balloon system (aortic and ventricular cusps 130, 131 and vertical segments 135) such that the port is simply a conduit defined by the balloon itself. Thus, when the balloon inflates, the central hollow area of the port 142 extends out to the tip of the valve leaflet and opens up for operative communication with the pocket 138.

The working port exit 140 is a terminal end of port 142 that extends radially from the distal catheter body 102 through the through conduit 104. The port 142 may be oriented at an angle relative to the central longitudinal axis A of the catheter body 102, with the working port exit 142 oriented toward the second annular valve 114. The angle is dependent on the degree of inflation of the valve cusp enclosure 106. The greater the inflation of the valve cusp enclosure 106, the more horizontal (closer to 90 degrees) the port 142 will be relative to the catheter body 102. The port 142 is made of a material similar to the valve cusp enclosure 106, so as to be able to convert from a transport state to a deployed state, i.e., it is elastic and will be stretched via attachment to the valve cusp enclosure 106. The port can be about 12F to 18F dimensionally and have a length in a range of 0.5 cm to 2.5 cm.

The walls of the port 142 can include inflatable chambers to receive a fluid to aid in deployment of the port. In another embodiment, the port 142 is constructed of concentric telescoping tubes 156 that can extend passively with expansion of the valve cusp enclosure 106, i.e., an attachment thereto. In any of the embodiment, the wall of the port 142 can include lumen or conduits in operative communication with the valve cusp enclosure 106, sensors position at the working port exit 140 or in the wall defining the pocket 138 of the valve cusp enclosure 106. The wall of the port 142 can include a plurality of layers, in any number suitable to provide an adequate number of lumen and/or conduits. In some embodiment, the wall of the port 142 include two to 10 layers of material.

In another embodiment, the port 142 includes high tensile, light weight titanium and/or plastic multi-linked multi-rotational prongs 157 attached along the exterior surface thereof or integrated into a wall of the port. The prongs can lengthen or shorten the port 142 in increments of microns and move the port directionally in X—Y—Z axes in increments of 1/10 of a degree up to increments of 1 degree.

As best shown in the comparison of FIGS. 1 and 2, the port 142 is expandable and collapsible between a deployed state (FIG. 2) and a transport state (FIG. 1), respectively. As shown in FIG. 3, the first ventricular cusp and aortic cusp pair 132a is configured to receive a single AVC, and via the fluid-tight engagement of the pair 132a with the AVV, the port 142 is used to remove blood from the pocket to facilitate repair of the AVC in a bloodless field. Tools can then be advanced through the catheter body 102, through the port 142, into the pocket 138 to treat the AVC. Since this treatment segment treats a single AVC, after treatment of a first AVC, the valve cusp enclosure 106 can be returned to a transport state, rotated, and re-deployed to the deployed state with a different AVC positioned in the pocket 138.

Still referring to FIGS. 2-3, the valve cusp enclosure 106 includes a secondary ventricular cusp and aortic cusp pair 132b, having a secondary ventricular cusp 130 and a secondary aortic cusp 132, which can define a pocket 138 to isolate a second and/or third valve cusp or are collectively shaped and/or inflated to lift one or more of the AVCs away from the ventricle toward the aortic wall during the treatment procedure. When there is a second pocket 139, the second pocket 139 will counterbalance the treatment cusp assembly and prevent dislodging thereof in the opposite direction. The pocket 139 may be made deep by inflating the aortic and ventricular cusps more 130, 131 relative to the vertical segments 135, thus leaving the two untreated leaflets (or one cusp if this is a bicuspid aortic valve) undeformed. This is especially important when the untreated valve cusps are heavily calcified and ankylosed and will need treatment also.

Generally, the ventricle cusps 130, in the expanded, deployed state, protrude radially outward from the through conduit 104 and are angled toward the ventricle. The pocket surface 144 defined by the ventricle cusps 130 are concave. The concavity can generally match the shape of the inferior surface of the AVC. The aortic cusps 131, in the expanded, deployed state, protrude radially outward from the though conduit and are angled toward the ventricle. The angle of the aortic cusps 131 relative to a central longitudinal axis A of the catheter and the radial outmost surface 146 of the aortic cusp is an obtuse angle Θ, which keeps the valve cusp enclosure 106 from blocking the coronary arteries and is at an angle β relative to the exterior surface of the through conduit 104. The angle β can be in a range of 10 to 60 degrees for the superior surface of each cusp, sometimes 30 to 60 degrees. The pocket surface 145 defined by the first aortic cusp 131 is contoured to have a convex portion and a concave portion. The contour of the pocket surface 145 is generally opposite of the shape of the superior surface of the AVC. The free end 136 of each cusp of the valve cusp enclosure 106 is expandable circumferentially, i.e., the cusp fans out to engage and cover the base of the AVC at the AVV.

The treatment segment 100 may be removably, replaceably secured to the catheter body 102. The connection can have an interlocking mechanism for retention of the treatment segment 100. This feature facilitates changing the treatment segment for one of a different size, specifications, or sending capabilities.

In an embodiment where the coronary artery ostia are more proximal to the aortic valve in the sinus of Valsalva, the degree of inflation of the first and second superior or cranial or proximal segments of the balloon of the valve cusp enclosure 106 will determine the angle and balloon surface's proximity to the coronary ostium. The exterior surface of the valve cusp enclosure 106 can flexibly bulge inward in a convex manner with the weight of blood in the proximal aorta during diastole by preferentially creating reduction in internal pressure in the balloon segment during systole to allow blood to pool in this area and returning the pressure back up during diastole so as to push the blood in the direction of the coronary artery cusp. This creates a current of blood flow from the aorta directed into the ostium of the coronary artery thus improving coronary perfusion during diastole while increasing the volume of blood available for the coronary artery filling pressure during systole.

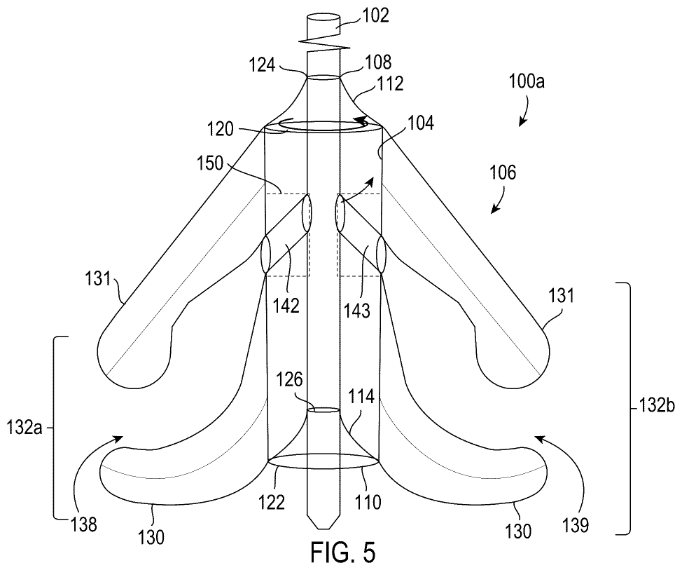

Referring now to FIG. 5, in another embodiment, the treatment segment 100a can have a first and a second ventricular cusp and aortic cusp pair 132a, 132b, which each have a port 142, 143, respectively, as described above and define a pocket 138, 139, respectively, as described above. Here, there would be four balloons, one each for each cusp. All other features being the same or similar and having like reference numbers, the description of which does not require duplication. Alternately, the illustration of FIG. 5 can represent a single enlarged pair 132a, which is sized to receive all AVCs at the same time, each positioned proximate one of the ports 142, 143. The AVCS can be treated simultaneously or sequentially. Alternately, in FIG. 5, port 142 can represent a first position of the port and port 143 a second position of the port, the port section 150 represented by the dashed lines being rotatable about the catheter body 102 to align with second AVC for treatment thereof.

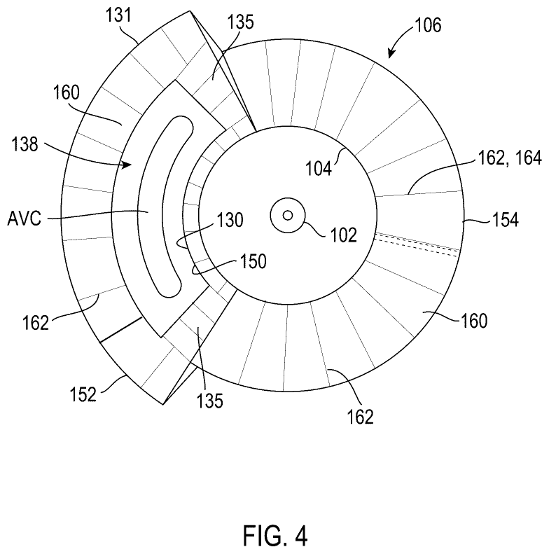

FIGS. 1-5 present a valve cusp enclosure 106 that has is inflatable balloon system. The one or more ventricular cusp and aortic cusp pairs 132a, 132b can each comprise a plurality of individually controlled inflation chambers, which are in fluid communication with a fluid source via the catheter body. The fluid source may be a liquid, such as saline, or other suitable, biocompatible liquid. The aortic cusp 131 of the one or more ventricular cusp and aortic cusp pairs is controllably inflatable to maintain blood flow to the right and left coronary arteries, i.e., when inflated it does not block the right and left coronary arteries as shown in FIG. 3 and discussed above. This allows fresh blood to flow into the coronary arteries in an unimpeded manner after it has been ejected out of the proximal tip of the proximal valve with each heartbeat into the aortic root and the sinus of Valsalva while also still maintaining runoff into the coronary arteries during diastole (as is physiologically present in normal coronary circulation) thus allowing full perfusion of the coronary arterial system. The plurality of individually controlled inflation chambers can run longitudinally (see FIG. 4, radially outward (see FIG. 2), and a combination thereof, and inflation of each can be controlled by an operator. The inflation can be accomplished simultaneously or sequentially relative to any number of the plurality of individually controlled inflation chambers.

Referring to FIG. 2 and FIG. 4, the ballon system can be described as having a first balloon 150 defining the first ventricle cusp 130a, a second ballon 152 defining the first aortic cusp 131a, and a third ballon 154 defining both the secondary ventricle cusp 130b and secondary aortic cusp 131b. Each balloon 150, 152, 154 has a plurality of internal chambers 160 separated from one another by internal walls 162. The plurality of internal chambers 160 are inflatable to differing pressures, thereby, when inflated partially or fully, the balloon has a three-dimensional shape to form a pocket 138 or to deflect an AVC as shown in FIG. 3. Each balloon can have any number of chambers. Each ballon 150, 152, 154 can include a manifold 164, which may be shaped as a rib, running longitudinally from the attached end 134 to the fee end 136. This can be generally centrally positioned in each ballon. Each manifold 164 includes a plurality of valves 166 and optionally conduits 168, one each in fluid communication an individual chamber 160 of the respective ballon. The valves 166 of the most proximal chamber(s) of each ballon 150, 152, 154 are set to a first opening and closing pressure. The valves 166 moving from the proximal chambers to the distal chambers closest to the free end 136 have respectively, second, third, fourth, nth opening and closing pressure, which are each different such that the first opening and closing pressure is lower, than the second, which is lower than the third, etc. for each balloon to inflate in a concentric, proximal to distal manner.

The third balloon 154, which is the largest of the three balloons has a plurality of horizontal chambers 160 in stacked relationship, like floors of a high-rise building, and has a plurality of vertical walls 163 (FIG. 4) within that are concentrically arranged therein, like apartments in the high-rise building. It can also have walls that are perpendicular to the concentric walls and thus form many cubical cells at each vertical level of all the segments of the balloon. Still referring to FIG. 2, this balloon 154 can have a multi-level manifold 170 having valves 172 in fluid communication with conduits, one each in communication with a single chamber. The pressures can be preset as described above to open the ballon proximal to distal. In another embodiment, the pressures for opening and closing the valves can be the same for simultaneously inflating and deflating the plurality of chambers.

In all embodiment, each chamber 160 of each balloon can have a pressure sensor, stretch receptor (embedded in a wall of each chamber), and/or a pressure transducer in electrical communication with an operating system for individualized control of the inflation thereof to a desired volume monitored and adjusted in real time by the operator or via an AI system and/or robotic system. This provides the advantage of control of the inflation of the distal most chambers to provide the desired pressure for the fluid tight seal against the endothelium or aortic annulus. It also allows for variable inflation of various chambers/segments to account for anatomic variation in a patient's vascular structure.

In one embodiment, the balloon chambers can be arranged as radially arranged inflatable layers that fill with fluid from the innermost to the outermost.

The inflation of the ballon system can begin from the distal end and progress to the proximal end, or vice versa, as well as from inner most to outermost in the radial direction. In the deployed state at the aortic valve the ballons are configured to acquire a size that is about half to two-thirds of the cross-sectional area of the natural aortic valve (and aortic root) and equal to or smaller than the aortic valve orifice it is sitting in.

The balloons can made of conventional balloon catheter materials or herein after developed materials. In another embodiment, layered cross-linked polytetrafluoroethylene (PTFE) membrane is used to make the balloons and internal segments of the balloons. Layered cross-linked polytetrafluoroethylene (LCL-PTFE) membrane is a thermoplastic polymer, elastic and highly flexible, solid at body temperature, self-lubricating with high strength and toughness, which is hydrophobic and radiation-resistant (it provides ultra-violet protection). Other possible materials include a light weight tightly woven nylon, synthetic silk, polypropylene, or extruded ultra-thin carbon fabric. LCL-PTFE membrane, when used to build the balloon will have varied thicknesses. The varied thicknesses depend on the elasticity property demanded from each aspect of the balloon.

Turning now to FIGS. 6, an embodiment of the treatment segment is shown with some additional features and is represented herein as treatment segment 100b. The first additional feature is a funnel-shaped entrance 190 at the distal end 110 of the through conduit 104 formed upon deployment and expansion of the through conduit 104 and/or the valve cusp enclosure 106. The distal end 110 includes expansion means 192 integrated into an elastic polymer material to form the funnel-shaped entrance 190. The expansion means 192 can be a plurality as circumferential elastic rings spaced apart a distance relative to the longitudinal axis A within an elastic polymer material, wherein the elasticity of the rings increases as the rings progress toward most-distal. The expansion means 192 can have frustoconical layers that slide against each other to elongate and form the funnel-shaped entrance. The expansion means 192 can be a material than can elongate or change shape in response to a signal, such as a thermal, optical, or electric signal. One examples is shape memory material, which can be a metal or a polymer. The shape memory material can be in the shape of circumferential rings as noted above or positioned similarly to a framework of an umbrella. In another embodiment, a spring-loaded terminal ring 194 defines the mouth of the funnel-shaped entrance 190, which can be expanded at deployment of the treatment segment 100b and retracted post-treatment. The spring-loaded terminal ring 194 can be made of metal, such as titanium or a titanium-magnesium alloy.

The expansion means 192 can also be a plurality of hinged titanium or a titanium-magnesium alloy rods each having at least two segments connected by a hinge. In this embodiment, a proximal rod 195 is hinged to an inner ring 196 that is secured to the exterior of the catheter body 102 and a distal rod 197 is hinged at its first end to the proximal rod opposite the inner ring and at its second end to an outer ring 194 (which can be elastic or are spring loaded). The distal and proximal rods 195, 197 fold proximally and are stored inside the balloon material in the transport state. Any and all of these funnel-shaped entrances, in particular, the expansion means 192 can also, because of its position, support the ventricular cusp(s) 130.

Still referring to FIG. 6, any of these expansion means 192 can also be present at the distal end 120 of the first annular valve 112 to assist the through conduit 104 in expanding during deployment of the treatment segment 100b, as represented by the dashed rods and hinges. The expansion of the first and second annual valves 112, 114 are linked to the expansion of the ballon system of the valve cusp enclosure 106 in an incrementally proportionate manner. In one embodiment, when the balloons system expands with introduction of a fluid media, the first and second annular valves 112, 144 can include mechanical means that expand based on robotic assistance (computer-controlled movements by introduction of electrical, thermal, or other forms of energy to the mechanical means.

A second additional feature shown in FIG. 6 is a mesh or sieve-like material 198 extending from the proximal end 124, 126 of one or both of the first annular valve 112 and the second annular valve 114. The mesh or sieve-like material 198 may have an axial length of about 2 cm to about 5 cm and can have elastic properties enabling the material to close against the catheter body and then open away therefrom during blood flow through the through conduit 104. The mesh or sieve-like material 198 is present to filter the blood to prevent blood clots from flowing into the aorta.

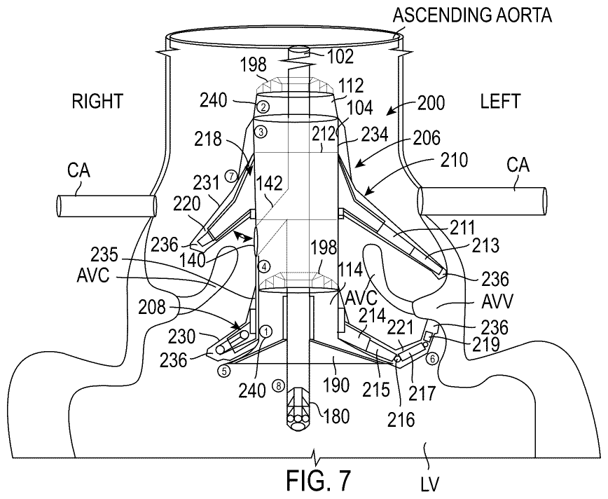

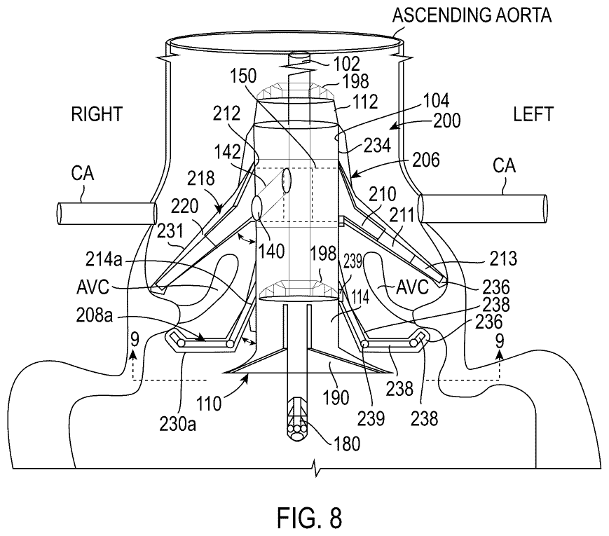

Turning now to FIGS. 7-9, a different embodiment for the valve cusp enclosure, represented here by reference 206, of an alternate style treatment segment 200 is disclosed. The treatment segment shown in FIG. 7 is a partially deployed state and comprises the additional features described in FIG. 6. The features that are the same or similar to FIG. 6 have the same reference numbers and are as described above. The treatment segment 200 shown in FIG. 8 is in a fully deployed state.

Referring to the aortic cusp 231 of the valve cusp enclosure 206 of both of FIGS. 7 and 8, each include a mechanical skeletal structure 218 that has a plurality of radially outward and ventricularly angled expandable arm segments 210 extending from a central hub 212 seated on and expandable and contractable with the through conduit 104. In one embodiment, the central hub 212 is made of a thermally activatable material enabling expansion and contraction thereof in response to thermal energy (increase and decrease, respectively). The expansion and contraction of the central hub 212 and the expansion and contraction of the arm segments 210, 220 are robotically controlled by operative communication between each and the printed circuit board in the terminal cap and/or an external operating system. The robotic control enables simultaneous and/or independent expansion and control of the central hub and any and all arm segments.

The mechanical skeletal structure 218 is located inside an annular sheath 220 that is fixedly attached to the treatment segment with a fluid tight connection, i.e., the attached end 234. The annular sheath 220 is formed of a material that has elastic and/or plasticity properties and an appropriate texture to engage the annulus of the aortic valved for a fluid tight seal. The material can include a magnesium alloy rubber or plastic. The elasticity and/or plasticity property is necessary for the expandable arm segments to be deployed radially outward. In one embodiment, the sheath is formed of LCL-PTFE membrane. The LCL-PTFE membrane can be pulled over or extruded over the skeletal structure 208, 218 of each cusp (single, double, or triple cusps) depending on whether the cusps are designed for single valve coverage, bicuspid valve coverage or coverage over all three valves and fixedly attached to the catheter body. In another embodiment, the LCL-PTFE membrane (superior and inferior) is continuous between the arms of the skeletal structure to create a webbed effect (like the feet of a duck). Sensors, wiring, and other electronics can be built into the LCL-PTFE membrane using a multi-material 3D additive printing process.

Other components of the treatment segment can be made of or coated/surfaced with LCL-PTFE. LCL-PTFE provides a smooth glassy surface that reduces friction between the surface of the catheter or component thereof and red blood cells, which reduces the likelihood of mechanical destruction of the red blood cells and traumatic hemolysis.

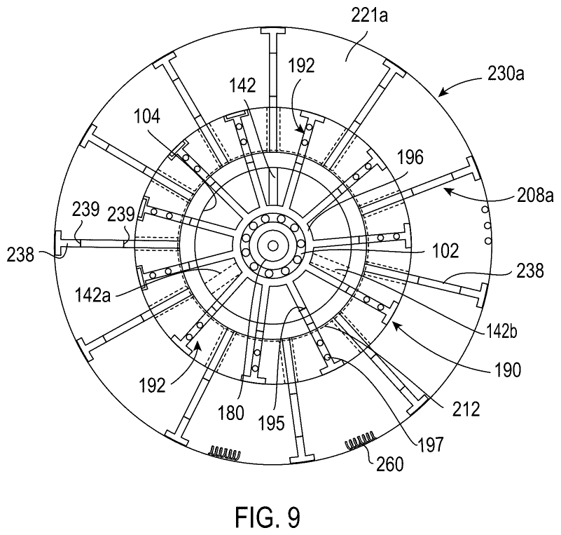

During deployment, a first arm segment 211 is advance from the expandable arm segment 210, and a second arm segment 213 is advanced from the first arm segment 211. These are slidingly telescoping segments nested one inside the other. The terminal end of the annual sheath 220 can be spring loaded between each of the expandable arm segments, so that deployment thereof, in particular, the second arm segment 213, spreads the sheath into its fully circumferential configuration, such as that shown for the ventricle cusp 230 in FIG. 9. The spring-loaded feature of the aortic cusp 231 or the ventricle cusp 230 can be a result of the elasticity of the sheath material 220 itself or springs 260 can be present as represented in FIG. 9. The expansion and retraction of the expandable arm segments 210 can be by wired mechanical connections or a result of shape memory material forming the arm segments 211, 213. The expansion of the expandable arm segments 210 can extend the aortic cusp 231 to two cm to six cm.

Still referring to FIG. 7, the ventricle cusp 230 of the valve cusp enclosure 206 of include a mechanical skeletal structure 208 that has a plurality of radially outward and ventricularly angled expandable arm segments 214 extending from a central hub seated on and expandable and contractable with the through conduit 104. In one embodiment, the central hub is the same as hub 212 or is independent therefrom and is made of a thermally activatable material enabling expansion and contraction thereof in response to thermal energy (increase and decrease, respectively). The expansion and contraction of the central hub and the expansion and contraction of the arm segments 214 are robotically controlled by operative communication between each and the printed circuit board in the terminal cap and/or an external operating system. The robotic control enables simultaneous and/or independent expansion and control of the central hub and any and all arm segments. The robotic control enables expansion or contraction of any of the cusps in increments of 1 μm to 10 μm.

The mechanical skeletal structure 208 is located inside an annular sheath 221 that is fixedly attached to the treatment segment with a fluid tight connection, i.e., the attached end 235. The annular sheath 221 is formed of the same material described above for the aortic sheath. The expandable arm segment 214 includes a first arm segment 215 that is advanced therefrom. The first arm segment 215 has a plurality of hinges 215 operatively coupling secondary segments 217 and 219, respectively, thereto to for a single elongate arm. The terminal secondary segment 219 rotates about its one of the plurality of hinges 215 to hold the terminal end 236 of the annular sheath 221 in engagement with the ventricle side of the annuls of the aortic valve. The first arm segment 215 can be slidingly telescoping relative to the expandable arm segment 214, i.e., it can be nested inside the expandable arm segment 214. The terminal end of the annual sheath 220 can be spring loaded between each of the expandable arm segments, so that deployment of the secondary arm segments 217 and 219 spreads the sheath into its fully circumferential configuration, as shown for the ventricle cusp 230 in FIG. 9. The spring-loaded feature is as describe above. The expansion and retraction of the expandable arm segments 214 can be by wired mechanical connections or a result of shape memory material forming the first arm segment 215. As illustrated in FIG. 9, the expandable arm segments 214 of the skeletal structure 208 can be placed at various intervals circumferentially to regulate the radius and cross-sectional area of the valve cusp enclosure 206.

Referring again to FIG. 7, a plurality of sensors 240, especially blood pressure sensors, are positioned on the exterior of the ventricular cusp 230 in the ventricle, on the exterior of the aortic cusp 231 generally proximate the coronary artery(ies), inside the through port 104, in the first annular valve 112, in the second annular valve 114, at the cap 180, and at the entrance to the second annular valve. Seven pressure sensors are illustrated in FIG. 7 as Circles that either have a number therein or a “P” for pressure. While seven sensors are shown, there can any number of sensors at myriad positions. These sensors may relay measure and transmit any information required for diagnostics and therapeutics. The sensors may function individually or collectively to create data points related to arterial lumen, ventricle, electrical data, lipid content of endothelium, blood pH, chemistry, oxygen levels, ultrasonic data (a few non-limiting examples). The pressure sensors measure systolic and diastolic blood pressure and send the data to the PCB in the cap 180 and to the external operating system. The data is used to regulate the amount of opening of the first and second annular valves 112, 114; thus, ensuring that the systemic BP is maintained to pre-procedure levels and that the blood pressure inside the through conduit 104, between the two annular valves, is maintained to a steady level.

Still referring to FIG. 7, the secondary segments 215, can be spring loaded, to help maintain engagement with the annulus of the aorta valve and to facilitates pulsatile fore and aft movement of the annular ventricle cusp 230, thereby adjusting for systolic and diastolic variations in the diameter of the ventricular side and aortic side of the outflow tract. Likewise, the arm segments 211 and 213 of the annular aortic cusp 231 can be spring loaded also. During ventricular systole, the expandable arm segments 214 proximate to accommodate the shortening and narrowing of the left ventricle outflow tract and vice-versa during diastole.

Turning now to FIG. 8, the valve cusp enclosure 206 has an annular aortic cusp 231 that is the same as that shown in FIG. 7. The port 140 can be on a rotatable hub 150 as described above with respect to FIG. 5. The annular ventricle cusp 230a is different than the one in FIG. 7, in that no telescoping segments are present. Instead, the skeletal structure 208a has a plurality of circumferentially spaced apart expandable arms 214a that each have a plurality of hingedly-connected segments 238 (hinges 239) that define an elongate arm. The plurality of segments 238 can be spring loaded for engagement with the annulus of the aortic valve as described above. FIG. 9 shows the annular ventricle cusp 230a and the funnel-shaped entrance 190 at the distal end 110 of the through conduit 104 in a deployed state. The sheath 221a can have the same features and is made of the same materials discussed above.

In both embodiments disclosed in FIGS. 7-9, the skeletal structure 213 and the terminal ends 236 of the aortic cusps 231, collectively may be referred to as “feet.” The deployment angle of each foot is individually adjustable based upon location of individual coronary artery ostia. The feet 213 and terminal end 236 can be directed to the wall of the Sinus of Valsalva either closer to the annulus or even in proximity to the coronary artery ostium. Coronary ostial perfusion can be increased by the feet of the aortic cusp immediately distal to the coronary ostium so as to create directional blood flow to the coronary artery.

The sheath covering the skeletal structure in FIGS. 7-9 can include or define a plurality of chambers that are inflatable. The fluid used to fill the chambers can be the same as described above for the balloon system. In one embodiment, the skeletal structure is robotically deployed first and then fluid is introduced to the chambers. In another embodiment, fluid is introduced to the chambers first and the robotics is used for finer adjustments and positioning of the feet. When chambers are present in the sheath, the chambers can be segmented with valve-controlled fluid communication therebetween. The manifold and valve system described for the ballon system above can be present here also. In one embodiment, compartments organized in a linear fashion from proximal the catheter body to distal from the catheter body include valves therebetween then open sequentially under preselected pressure so that the chambers fill first proximally and last distally.

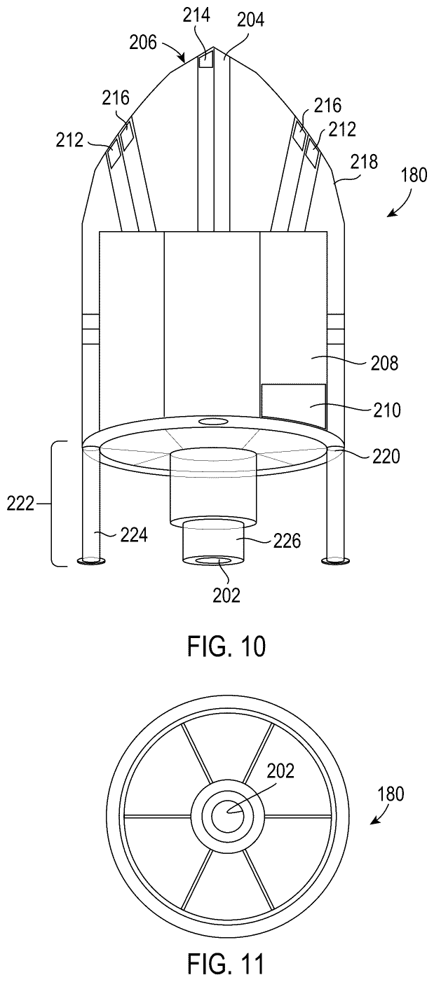

Turning now to FIGS. 10 and 11, enlarged views of a cap 180 are provided to better exemplify features thereof. The cap 180 has a working channel 202 having an open end 204 at the terminal end 206 of the cap 180 to allow egress and ingress of a tool (not shown). Prior to deployment of a tool, the cap 180 is closed, thereby blood or other fluids do not ingress or egress through the cap 180. Rather blood or other fluids flow around the cap 180 to the second annular valve 114. The tool can be inserted into the ventricle and advanced to the left atrium and even through the mitral valve, if desired. In one embodiment, the tool includes sensors to measure blood pressure, blood flow (ultrasound), EKG electrical activity and can transmit electrical stimulus to the atrium or the ventricle to generate pacemaker functionality. In another embodiment, the tool can perform therapeutics such as thermal (ablation), cold ablation, closing a hole(s) in the heart (ASD or VSD), apply grafting materials, build electrical networks in the heart muscle of ventricle or atrium, pass balloon to pulsate blood in the atrium, snare atrial clots or implant umbrella catheters to prevent clots or strokes.

The diameter of the cap 180 is preferably in a range of 12F to 18F and has a length of about 0.5 cm to 3 cm. The cap 180 terminates with a conical tip 218 that has a smooth curved dome-shaped. The cap 180 has a base 220 that defines a plug 220 receivable in the terminal end of the catheter body. The plug 220 include electrical terminals 224 and working channel connectors 226 for communication between the catheter, an external operating system, and the equipment and/or printed circuit board 208 and power source 210. The cap 180 may be built from biocompatible materials. Some example materials include polymers, titanium, aluminum, magnesium, silver, and alloys containing any one or more of these metals or a composite containing any one or more of these materials. Additional details about the operating system and connection to the catheter body is disclosed in co-pending U.S. application Ser. No. 17/815,282.

The cap 180 encloses a printed circuit board 208 operatively connected to an enclosed power source 210. The printed circuit board 208 can have any and all features described in co-pending U.S. Application No. 63/494,799 and 63/494,800. The power source 210 can be a battery, which can be a pre-charged, rechargeable, or continuously charged by an external power source through wires built into the wall of the catheter body 102. The power source 210 can power any equipment carried or built into the cap 180. The equipment can include (i) sensors 212, such as intravascular ultrasound (IVUS), capacitive micromachined ultrasonic transducers (CMUT), infra-red sensors, oxygen sensors, blood pressure sensors, etc., (ii) imaging 214, such as fiberoptics, cameras, etc. as individual items or as arrays, and (iii) emitters 216, such as LEDs, infra-red, or other light sources. Dual illumination modalities or multi-modal illumination offers optimal illumination of vasculature, lumens, or cardia imaging, i.e., LEDs and infra-red emitters are both present, which can assist in performing intra-cardiac surgical repair of the ventricle, atrium, and valves in-situ (thus, obliterating the need for trans-thoracic surgery). Endo-myocardial reinforcement with Teflon/Dacron or other materials or electrical wiring can be embedded in the myocardium to strengthen the heart muscle and provide electrical stimulation.

The equipment is electrically and operatively in communication with the printed circuit board 208 to provide a feedback loop for an operator or computer guided positioning of the catheter and treatment segment 100, imaging the aortic valve, sensing pressures in the ventricle and aorta, providing continuous EKG recordings and pressure dynamics with ventricular outflow and aortic blood flow and coronary artery flow. The communication can be wired or wireless, and when wireless can include Bluetooth two-way communication and can include WIFI capabilities. Geo-positioning can be included for transportation of the treatment segment 100 to the aortic valve and for proper positioning for deployment thereof. Geo-positioning can also be used to locate electrical abnormalities in the atrium or the ventricle and map problem areas for transcatheter ablation and for rebuilding innate electrical circuits or implant electrical circuits. This could take the place of an implantable pacemaker.

All data transmitted and received via the cap 180 can be recorded in Blockchain using NFT to build non-destructible data sequence of events for the purpose of recording and machine learning. AI or Machine Learning programs assists robotics through IOT (Internet of Things) referenced anatomical accuracy in the vascular tree of a given subject. IOT referenced GPS is accurate to the level of a nanometer when assisted by fiberoptic camera illuminated with IR/cold LED and ultrasonically guided by IVUS/CMUT present in the cap 180.

In one embodiment, the cap 180 includes a fiberoptic camera system, which may include one fiberoptic camera filament or multiple radial systems of fiberoptic filaments to capture a 360-180-degree view of the vasculature. The fiberoptic camera system captures images and/or video in real time and is ultimately connected to a display such as a computer screen through a wired or wireless connection.

The sensor in the cap 180 transmit data to the printed circuit board 208, to the catheter, to the catheter's operating system, which is typically external to the patient, and to any robotic system included in the catheter and/or treatment segment 100. The data can be used for myriad functions, including robotics navigation, robotic deployment of the treatment segment, and AI systems involved in navigation, placement, and/or treatment of the aortic valve cusps. Systolic and diastolic BP, blood temperature, and oxygen saturation assists in optimizing the centralized location of the catheter tip inside vasculature. This method prevents dissections, plaque rupture and perforation. EKG recording is important to regulate and time the opening and closing of the proximal end of the first annular valve 112 and the proximal end of the second annular valve 114. When QRS complex of the EKG is arrived at, robotics can assist dilation of the proximal ends of the first and second annular valve 112, 114 in the embodiments of FIGS. 7-9 while the onset of T wave brings about gradual closure of the proximal ends. The EKG-synchronized opening and closing of the first and second annular valves 112, 114 maintains stable blood pressure inside the treatment segment 100, thus maintaining a stable diameter of the entire catheter. The stability of the catheter cross-sectional dimension maintains stable positioning of the valve cusp enclosure 106, thus stabilizing positions of all other processes that occur related to valve cusp remodeling. The prevention of oscillation of the entire assembly that naturally occurs during systole and diastole of the heart creates a stable platform for all robotically assisted remodeling processes.

Still referring to FIGS. 10-11, continuous BP recording provides additional information regarding left ventricle outflow tract pressures. This helps the synchronization of inflation of the valve cusp enclosure 106. As the valve cusp enclosure 106 gradually inflates or robotically expands, the blood flow through the through conduit 104 incrementally increases as the blood flow around the catheter and balloons gradually decrease. One goal is to maintain constant net blood flow and stroke volume into the aorta and stable blood pressures in the aortic root during the procedure, thus maintaining physiology similar to that the subject is accustomed. A second goal is to remodel the aortic valve to improve perfusion (to levels pre-aortic stenosis or aortic valve disease). The collective goals minimize or prevent damage to the brain and other organs seen with traditional systems that usually cause drop in blood pressure during procedures involving the aortic valve.

The catheter body 102 can be any commercially available catheter. The catheter body 102 is typically about 4 to about 6 feet in length and is made of traditional catheter materials, which can include polytetrafluoroethylene or cross-linked polyethylene, but can be any length necessary to reach a treatment site. The catheter body includes at the distal end, a treatment segment 100, 200, described herein, a main body or shaft that is inside the vasculature of the patient, and a proximal end connected to an operating system that is located outside the patient's body. The catheter body 102 has, running the internal length thereof, lumens of various dimensions and functions, at least one lumen is a delivery sheath through which any number of tools can be deployed to and through the port 142 for treatment of an aortic valve cusp. Additionally, electronics, fluid conduits, etc. can be built into the wall of the catheter body 102 rather than being inside a lumen. One lumen can be a fluid delivery tube in fluid communication with the balloons 150, 152, 154 and the manifolds 164 therein for delivering fluid to inflate the balloon or removal of fluid to deflate the balloon. The wall of the main body can have built in conduits, electrical wires, shape memory materials, and can define a plurality of lumen for communication with any aspect of the treatment segment, including the valve cusp enclosure, the terminal cap, any and all sensors, and other tools/equipment.

In one embodiment, the distil end of the catheter body most-proximate the cap 180 comprises a shape memory material, such as a thermally activated shape memory polymer in operative communication with electrical wires in the wall of the catheter body that can transmit heat to the thermally activated shape memory polymer. Thermal activation of this polymer adjusts the flexibility of the cap and/or distil end of the catheter body to impart a bend thereto for navigation of the catheter through curves in the vasculature.

The catheter can include a computer-communicative guidance system. The catheter wall(s) or lumen therein include a plurality of metal or metal alloy wires that have tensile strength and flexibility spaced apart about the circumference of the catheter. In one embodiment, the number of wires ranges from four to 36. These wires run at least the length of the treatment segment and terminate at the base of the cap 180. These wires enable the computer to track and perform navigation thereof by guiding a latitude of about 180 degrees in all directions. In one embodiment, data from the IVUS in the cap is communicated to the computer-system to create a visual display on a computer screen and function as a component of the guidance system. In another embodiment, the proximal end and the distal end of each of the first and second annular valves and other components of the treatment segment, including the terminal cap, can have an imprinted marker system that is configured to be in operative communication with a computer system as part of a computer-communicative guidance system. The imprinted marker system may communicate with the computer using Bluetooth emitting Wi-Fi configured blockchain enable NFT.

In another aspect, the catheter body 102, lumens therein, the exterior of the cap 180, or any other feature of the catheter that has a diameter can be constructed to has electromagnetically coupled bands or thermally activated bands (such as shape memory materials) spaced at intervals that are arranged in a manner to enable enlargement or contraction of the diameter thereof. In one embodiment, the intervals can be 0.1 μm to 5 μm, or larger such as 4 mm to 10 mm. Introduction of current or heat can activate the band to change the diameter of the respective feature. The catheter, its lumen and the side port may benefit from this feature in order to transport a large tool or instrument to the treatment site. This feature is meant for diameter change without any elongate of the respect feature. In other embodiment, however, the change in diameter occur along with elongation.

The treatment segment 100, 200 can include sensors and other electronic equipment in positions other than the cap 180, such as inside the through conduit 104 and either or both of the first and second annular valves 112, 114, on the exterior of the catheter body 102, on the exterior of the valve cusp enclosure 106, 206, in the wall(s) of the valve cusp enclosure that define the pocket 138, and even in the terminal ends 136, 236 of any lobe of the valve cusp enclosure 106, 206. The sensors can include a blood pressure sensor, infra-red sensors, EKG, etc. The electronic equipment can include imaging technologies, such as fiber optics, IVUS and/or CMUT, illuminated fiberoptic electron microscopy and guided biopsy. The electronic equipment can include illumination technologies, such as LEDs and infra-red emitters.

In one embodiment, blood flow sensors are placed on the exterior of the proximate the coronary arteries and proximate the left ventricle to measure the volume and pressure of blood flowing from left ventricle to the aorta. As the valve cusp enclosure is deployed, these sensors record changes in blood pressure and provide the data the onboard PCB of the cap and/or the external operating system so that the through conduit 104 and first and second valves 112, 114 are opened proportionally thereto. Sensors placed on exterior of the catheter, exterior of the valve cusp enclosure, and inside the through conduit and valves can measure flow rates/velocities, blood systolic/diastolic pressures, cross-sectional area during expansion and contraction of the valve cusp enclosure and the through conduit, oxygen concentration and temperature. The sensors help compare the results of ventricular stroke volume and pressures with aortic stroke volume and pressures and provide feedback to modulate the opening and closing of the first and second annular valves. This provides the patient/subject with adequate perfusion of all organs.

In any of the embodiment, each terminal end 136, 236 of a cusp of the valve cusp enclosure can include touch and/or pressure sensors therein. The data from such sensors can aid in ensuring adequate pressure for a fluid tight seal to the annulus of the aorta. The terminal ends 136, 236 can also include imaging and illuminating equipment to aid an operator in guiding the same into contact with the annulus of the aorta.

In any of the embodiment, the EKG technology and the data it provides can be used by the computer system(s) to open or close the first and second annular valves 112, 114 as needed to control the blood flow and blood pressure of the patient. Under some conditions, the two valves 112, 114 open simultaneously. Under other conditions as determined based on the EKG data, the second annular valve 114 is opened first and the first annular valve 112 is opened about 2 msec to 20 msec later. Under yet other conditions as determined based on the EKG data, the first annular valve 112 is opened first and the second annular valve is opened about 2 msec to 20 msec later.

In all embodiments, the imaging device can be any commercially available imaging device or hereinafter developed technology. In one embodiment, the imaging device is selected from near-infrared spectroscopy plus intravascular ultrasound, fiberoptics, or capacitive micromachine ultrasound transducer. Imaging will be in real time, thereby enabling the operator or computer to make decisions about balloon inflation, balloon size, and balloon positioning relative to a treatment site.

### Deployment of the Catheter

In one embodiment, a catheter with the treatment segment 100, which includes the terminal cap 180, is transported to the aortic valve over a guide wire, where once the distal and proximal valves 112, 114 are located at their appropriate positions, as described above, the expandable valve cusp enclosure 106 is expanded, and the guide wire can be withdrawn. In the deployed state, all sensors, imaging, and other electronics are activated, and two-way communication therewith is functional and confirmed. Data is recorded either internal or external to the treatment segment 100. All such communications can be wired or wireless. In one embodiment, such communication use WIFI, Blockchain and NFT's via an Internet of Things (IoT) and/or servers. As used herein, “Internet of Things” has its common ordinary meaning-devices with sensors, processing ability, software and other technologies that connect and exchange data with other devices and systems over the Internet or other communications networks.

In one embodiment, the distal end of the treatment segment is opened (possibly funnel-shaped) proportionally to inflation/deployment of the valve cusp enclosure. As the peri-catheter space begins to occlude due to inflation/deployment of the valve cusp enclosure, the through conduit dilates to take up the function of transferring blood flow into the aorta. The cap of the treatment segment measures systolic and diastolic blood pressure in the left ventricle outflow tract and the data is relayed to the computer system. Based on this data, the robotics open the proximal end of the second annular valve during systole timed to beginning of the QRS complex on EKG data.

Machine learning (AI) and the robotics use the data from above mentioned inputs to generate strategy for the entire procedure. Data is used to measure the topology of the native aortic valve, measure calcification, amount of narrowing and annular calcification and strength, thickness of cusps and other relevant data, to continuously modulate the amount of opening of the valve cusp enclosure 206, and to calibrate the amount of opening to match the patient's needs such as BP, volume of blood flow, and to match the size of pre-procedure aortic valve opening. The system has the capability to incrementally increase the internal size of the first and second annular valves 112, 114 and inter-valvular segment of valve cusp enclosure 206 to match the intra-procedure and post-procedure increasing size of the aortic valve as the modeling process makes the valve cusps more pliable and the stenotic aortic valve opens wider.

The ventricle cusp deploys first, which coincides with deployment of the first and second annular valves. The ventricle cusp deployment begins with robotic extension of a proximal segment first, then the middle segment and then the terminal segment of each arm of the skeleton structure of all arms simultaneously, which spreads the valve from center to periphery.

In the deployed state, examples being shown in FIGS. 3, 6-9, blood flow from the ventricle is fully conducted through the through conduit 104 and no blood is moving in peri-catheter region as it is sealed off by the valve cusp enclosure 106. Aortic blood pressure and cardiac output are now stable. Next, the bloodless field is created around the one or more aortic valve cusps being treated. Via the port 142, the pocket 138 formed by the valve cusp enclosure 106 is flushed and aspirated using an aspiration catheter. Saline or similar material is used to flush the space within the pocket 138. Imaging, such as fiberoptics, is used to confirm the successful creation of the bloodless field.

Once the bloodless field is created, the aortic valve can be treated and remodeled one cusp at a time. Each cusp can be treated and remodeled first on the aortic side and then on the ventricular side, or vice versa. The bloodless field gives the operator the freedom to treat and remodel a valve cusp and make simultaneous comparisons to the other cusps to ensure that the cusps will seat securely after remodeling is complete.

The tools used to treat the aortic valve cusps can be fed through the catheter body 102 individually or collectively to and through the port 142 into the pocket 138. The catheter body 102 may include therein one or more aspirators, imaging device, cutting tool, and other tools needed during the medical procedure. After completion of the treatment and administration of a drug treatment, if needed, the tools and balloon catheter are removed from the patient. Following the medical procedure, the medical professional may instruct the patient regarding a post-care regimen of drug treatments, activities, and the like.

An example tool is an aspiration and infusion system configured to infuse saline into the field of operation and aspirate the saline and flushed materials out of the field such as to eliminate blood, calcium, fibrinous material, and other tissue components broken down during treatment of the heart valve.

The method can include introduction of a tool configured to determine the lipid burden of the surfaces of the valve. The tool can assist in determining whether the lipid burden includes a high lipid burden, a low lipid burden, or a high to low lipid burden ratio. The tool can be a near-infrared spectroscopy plus intravascular ultrasound or a capacitive micromachine ultrasound transducer. These tools can also be used for imaging the valve and its surfaces. The capacitive micromachine ultrasound transducer is much smaller than other imaging devices.

Another example tool is a laser. The laser may be an ultraviolet laser, such as an excimer or exciplex laser. One commercially available laser is the ELCA™ laser from Phillips (FDA approved) adapted to fit the lumen utilized for delivery to the treatment site. When activated, the laser breaks calcium crystals, burns fibrous tissue on ventricular and aortic surfaces of cusps, and/or melts lipid material of each valve cusp. Each of these tasks may require a different wavelength for the laser; thus, the laser can have an adjustable wavelength. Alternately, different lasers can be transported to the treatment site through the lumen of the catheter. When cutting with the laser, gentle saline flush can be applied.

Another tool may be configured to deliver medications or products directly into the left ventricle or into the operable area of the active valve cusp. In another embodiment, the catheter itself or a tool carried thereby infuses medications or chemicals used for radiographic or other data acquisition, diagnostics, or therapeutics of the heart, coronary arteries, or other organs of the patient.

The illuminated fiberoptic electron microscopy and guided biopsy can be used to assist in-situ diagnosis of medical pathology like cancer, amyloidosis, fungus or bacterial vegetations on valve leaflets or endocardium. It can also be used to map extent of graft-vs-host rejection, endomyocardial biopsies etc., and other organ evaluations in a similar manner.

The balloon catheters disclosed herein and the various methods of use of such balloon catheters can be implemented to treat the aortic valve cusps in myriad ways, such as: changing the cross-sectional area of the aortic valve, treating aortic ankylosis, i.e., bring back flexibility and mobility to the cusps, remove calcifications from the cusps, replace and/or synthesize fibrosa epithelium on the aortic side of a cusp, replace and/or synthesis ventricularis epithelium on the ventricular side of the cusp, replace and/or synthesize spongiosa inside the body of the cusp, remove lipid material from the sub-epithelium of the cusp, and recreate the aortic valve anatomically, morphologically, and functionally.

In all embodiments disclosed herein, a coating of heparin, clopidogrel, sirolimus, or tacrolimus can be present to prevent blood from clotting along any and all surfaces of the treatment segment and/or catheter body 102, including the mesh or sieve-like material 198.

While the catheter herein is described for treating the aortic valve, it can be configured for treatment of other valves in the heart, other organ systems, and it can be made in a miniaturized version for functionality in other lumens, such as the coronary artery, other blood vessels, an in other organs, such as systems like the GI tract (small intestine, colon, stomach, pancreas), hepato-biliary system, urinary tract system, spinal cord, musculoskeletal and orthopedics, respiratory and pulmonary endoscopy and provide insight into diagnostics and provide therapeutics to include nano-therapeutics and theranostics in those systems. Oncology drug delivery may be possible through this system. The bloodless field created by this invention can be used in other systems and applications such as tumor management as well. The systems and methods disclosed herein can include AI computer-generated calculations for the cross-sectional area of a lumen of the aorta or of the ventricle. This is also applicable to any other lumen of the body. As described above, having capacity to recreate flow dynamics will assist in forecasting the cross-sectional area of the tube or lumen that will create laminar flow of fluids.

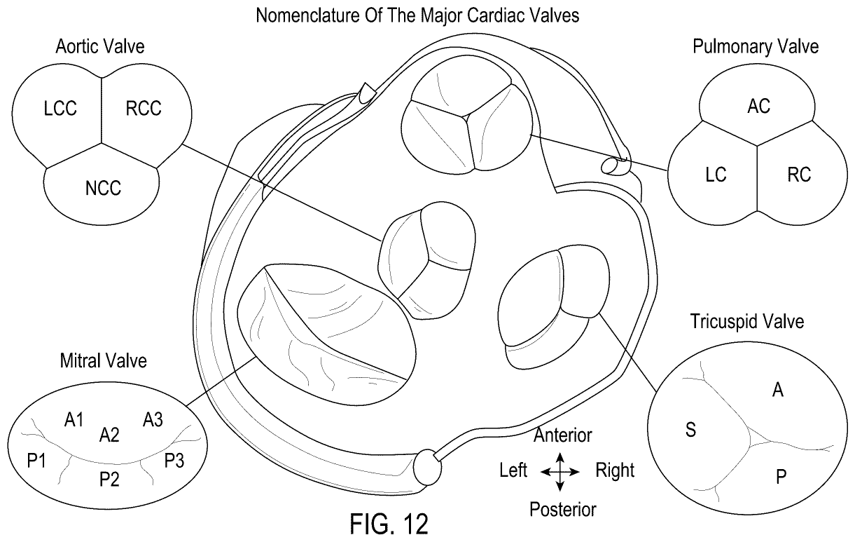

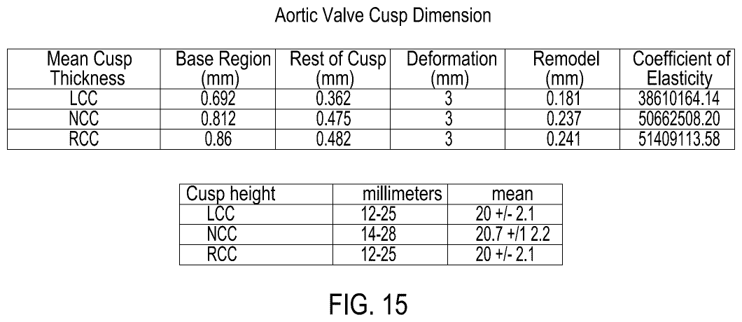

Referring now to FIG. 12, an illustration of the cusps of the aortic valve is provided including the abbreviation labels for the left coronary cusp (LCC), right coronary cusp (RCC), and non-coronary cusp (NCC). Dimensions for standard aortic valve cusps are provided in FIG. 15. The dimensions reflect that the thickness of the base of the cusp is typically twice that of the rest of the cusp.

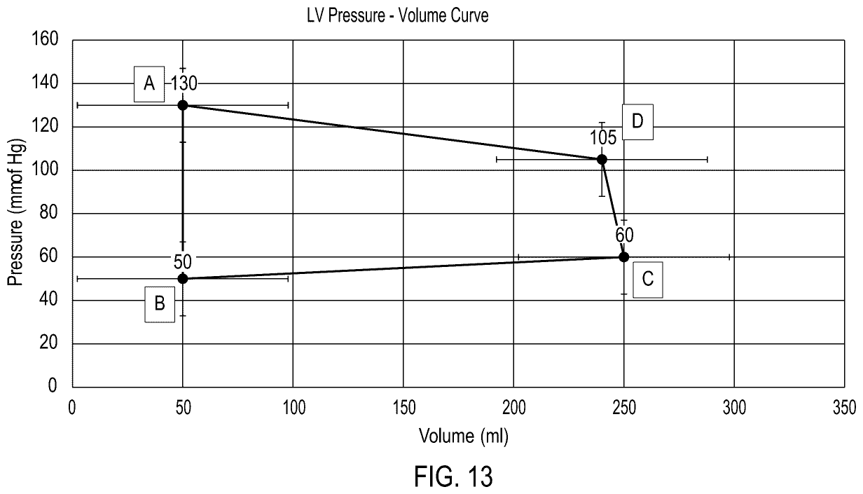

A left ventricular (LV) pressure-volume curve is provided as FIG. 13. The values presented therein are for descriptive, exemplary purposes only. The LV Pressure-Volume curve is a loop that starts at the end of systole with the closure of the Aortic Valve (A). The ventricle relaxes (diastole) and pressure drops while the mitral valve has not yet opened. The mitral valve opens (B) and blood fills the left ventricle. The pressure begins to increase and finally the ventricle fills up and begins the onset of systole (C). Mitral valve closes rapid contraction of LV increases its pressure till the Aortic Valve opens (D) and blood flows rapidly into the aorta and the ventricle empties out with the final contraction and then the Aortic Valve closes (A) and the cycle begins again. In the treatment methods disclosed herein, an LV Pressure-Volume curve is mapped into a computer by generating data of myriad cycles for a patient to create a mean and at least six standard deviations. This data will be used in modeling morphology and elastic properties for the patient's aortic valve cusps and therefrom implementing a treatment.

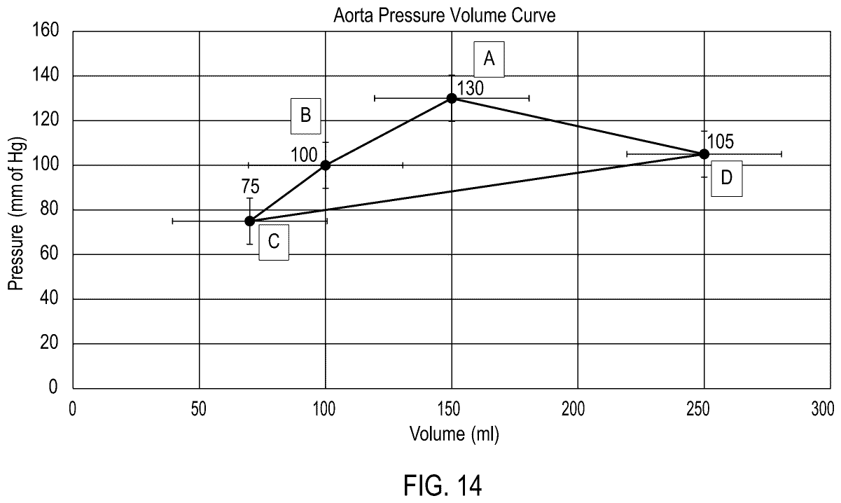

An aortic Pressure-Volume Curve is provided as FIG. 14. The values presented therein are for descriptive, exemplary purposes only. A-D correspond to the same systolic and diastolic events described above with respect to FIG. 13. Hereto, for the treatment methods, an aortic Pressure-Volume Curve is mapped into a computer by generating data of myriad cycles for a patient to create a mean and at least six standard deviations. A maxima and minima for the volume and pressures flowing across the aortic valve an into the aorta are determined. In a diseased state, such as aortic stenosis, there will be significant deviation from normal values. The purpose of mapping a patient's Pressure Volume Curves is to see the pathological pressure changes. This data will be used in modeling morphology and elastic properties for the patient's aortic valve cusps and therefrom implementing a treatment. The goal is to restore normal pressure-volume loops, which will help increase the longetivity of the modelled aortic valve and reduce long term risk of stroke, blindness and congestive heart failure.

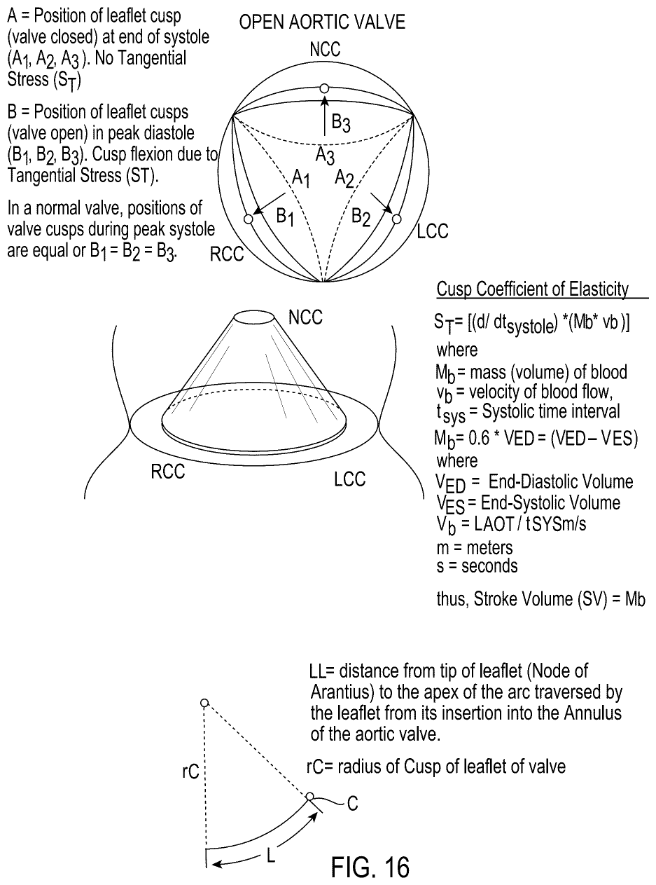

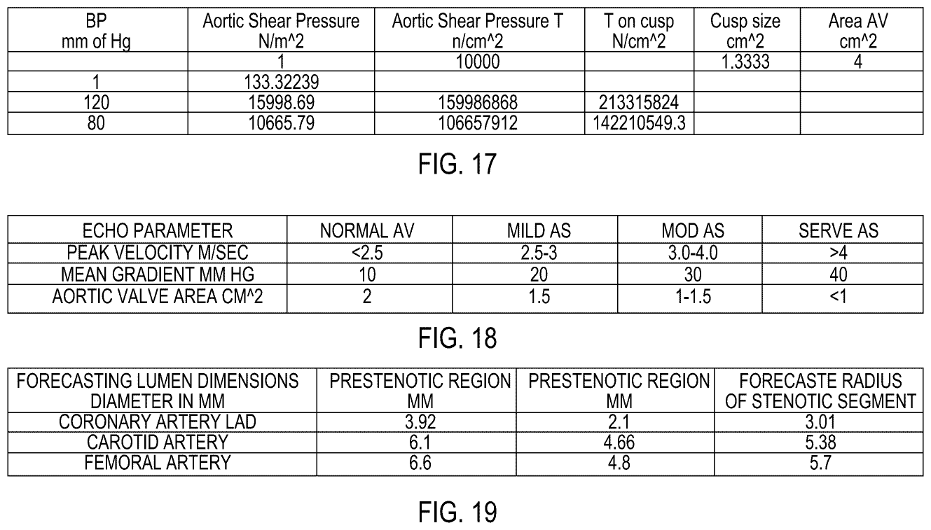

The method involves implementing algorithms to convert tangential shear (T), also known as Wall Shear Stress (WSS), into blood pressure in mm of Mercury (and vice versa) at a level of a narrowest cross-sectional area of the aortic valve in systole, based on values derived from the ventricular side of the valve and aortic side of the valve. T is then used to determine the pressure exerted by a column of blood (blood follows Newtonian principle of fluid) on the body of each aortic valve cusp surface and on the frame of the aortic valve cusp. Since all three cusps will be exposed to the same amount of T due to being at the same level in the path of blood coming from LV to Aorta, the assumption is that all three cusps will have same degree of blood pressure exerted on their surface areas. Referring to FIG. 16, the body of each cusp (RCC, LCC and NCC) will need to undergo equal amounts of deformation (A→B where B1=B2=B3) during ventricular systole to maximize the aortic valve cross-sectional area at its narrowest level or “smallest cross-sectional area” during systole). This must occur in response to rising and falling blood pressure from the LV outflow tract during systole such that the remodeled aortic valve provides the most efficient transfer of blood from the left ventricle to the aorta. This deformation is proportionate to the tangential shear stress created by the blood flow (following Newtonian Fluid principles and second law of motion). Values for T are calculated and presented in FIG. 17.

To create this deformation of the aortic valve cusp(s) remodelling will incrementally thicken the body of each diseased valve transversely with the thinnest portion in the center and the thickest portion adjacent to the cusp frame where the body of the cusp will end and the frame will begin. The amount of deformation will be the greatest in the center and almost absent at the periphery where the body is inserted into the annulus and the frame. The cusp require elasticity to move effectively, which is expressed herein as a cusp coefficient of elasticity. Still referring to FIG. 17, the cusp coefficient of elasticity (St) is given by the formula: St=d/dt(M*V). St is also the equal to the tangential stress T (or WSS) applied by a Newtonian fluid to a fluid-solid interface at a given velocity and viscosity of the fluid, dt is systolic time interval during which there is forward flow of blood from ventricle through the aortic valve to the aorta, M is the mass of blood moved and V is the velocity (1 m/s for a normal aortic valve) at which blood is moved. Assuming the rheological properties of blood remain constant (blood density=1060 Kg/m where pure water density=1000 kg/m), the mass of blood moved can be calculated based on volume (stroke volume per ventricular contraction) being an average of 95 ml (+/−14 ml). Mass=volume*density.

Normal cusp thickness is not the same throughout the cusp surface as discussed above with respect to FIG. 15. When remodeling is being performed, a LASER is used to thin down the aortic (spongiosa) and the ventricular (Ventricularis) calcified and fibrous lining down to the fibrosa layer. Then, the lining of the cusp is built back up, for example by 3D printing spongiosa, ventricularis, interstitium, or a combination thereof onto the cusp. These are printed to a minimum numbers of millimeters so as to restore the original minima and maxima of the cusp thickness and to maintain the coefficient of elasticity. About 3 mm of deformation is necessary at the center of each cusp and is about 3 times higher resistance to deformation (at least twice the thickness in the base region) at the periphery of the cusp where less or no deformation is required. The bases of the cusps also have adherence to the Aorto-ventricular junction or annulus.

The treatment method includes AI aided reconstruction of the Node or nodules of Arantius of one or more of the aortic valve cusps. The node a structure present on the tip of each cusp, which is flush with the ventricular surface and spearhead shaped on the aortic surface. The three nodules (one at the tip of each cusp) approximate to produce a cone effect when the valve is closed. This helps blood to slide down into the aortic side of the cusps and cause the cusps to sag like a tent. See FIG. 16. This sag allows better approximation of the cusp frames along commissural lines and creates a snug fit of the valve to prevent regurgitation of blood into the ventricle. The node is destroyed or undergoes hypertrophy due to aortic stenosis or post repair aortic incompetence. This will be remodeled using AI to precisely create the exact dimensions. The reconstructed node will be 1.2 mm-2.5 mm tall, 0.8 mm-1.2 mm thick at the base and span the two frames from the apex approximately 1.2 mm-1.4 mm wide. When all three cusp tips have a node, together they will form a dome like cap on the aortic side of the closed aortic valve in diastole. The node is known to prevent aortic insufficiency.

AI calculates the value of thickness of the cusp body at each incremental distance from the center of the cusp body based how much deformation (B) is needed to create the largest vale for the smallest cross-sectional area (SMCA). A LASER is used to thin down the calcified fibrotic valve thickness until all calcified and fibrotic material is removed. Then AI aided remodeling and 3D printing of tissue increases the thickness back to the normal thickness or slightly thicker valve cusps to withstand the shear pressure T. The tissue material for 3D printing can withstand/produce 1,752,000,000 cycles of cardiac contractions so as to reflect 100 beats per minute for 40 years. In one embodiment, the tissue material is a nanotechnology material, which can include elastin and/or stem cells. In another embodiment, the tissue material is a drug treatment that comprises collagen and/or carbon dots comprising stem cells, as disclosed in co-pending U.S. Application No. 63/494,794. When this drug treatment is use, the method further includes activate the collagen by application of an activating wavelength of energy.

Referring now to FIG. 18, in aortic stenosis the velocity of blood is 2.5-2.9 m/s in mild stenosis, 3.0-4.0 m/s in moderate stenosis and >4.0 m/s in severe aortic stenosis. The normal aortic valve diameter is 21 mm to 23 mm and thus normal SMCA of arotic valve is 3-4 cm² with <1 cm² being severe aortic stenosis.

The method will also include mapping a patient's anatomy of their aortic root, aortic valve and left ventricle using the features of the treatment segments 100 disclosed herein, especially features for imaging, mapping, and sensing present in the terminal cap 180. Such measurements will provide actual dimensions, pressures, flow dynamics and pressure gradients at the aortic valve of the patient. AI is then used to calculate ideal values for the patient post-remodeling of the aortic valve. AI forecasts ideal stroke volume, blood flow velocity, ideal SMCA, cusp dimensions and thickness, elasticity and tensile strength of the cusp body and frame. This information is used while perform remodeling of the aortic valves cusps for recreation of all normal valve dimensions during remodeling.

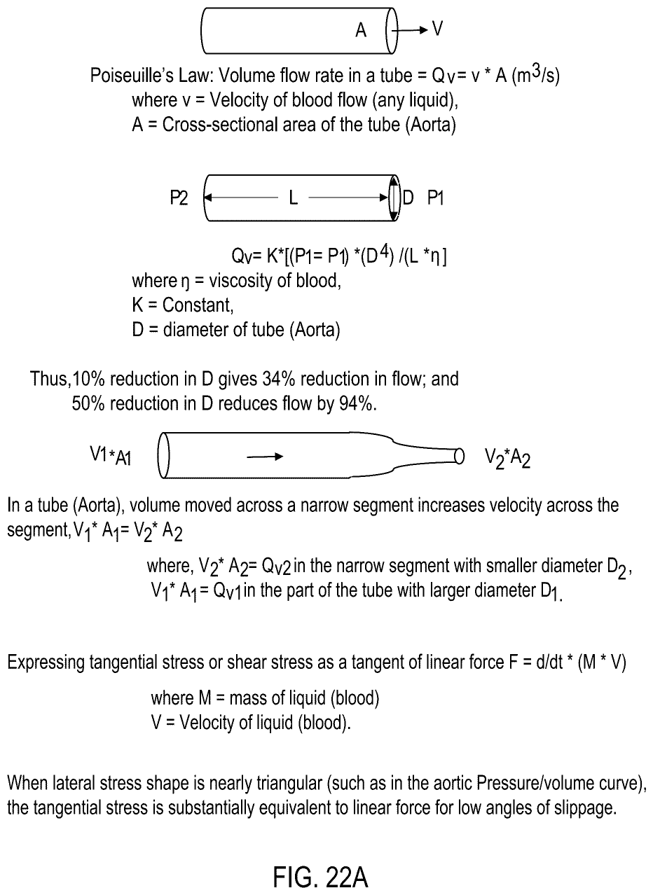

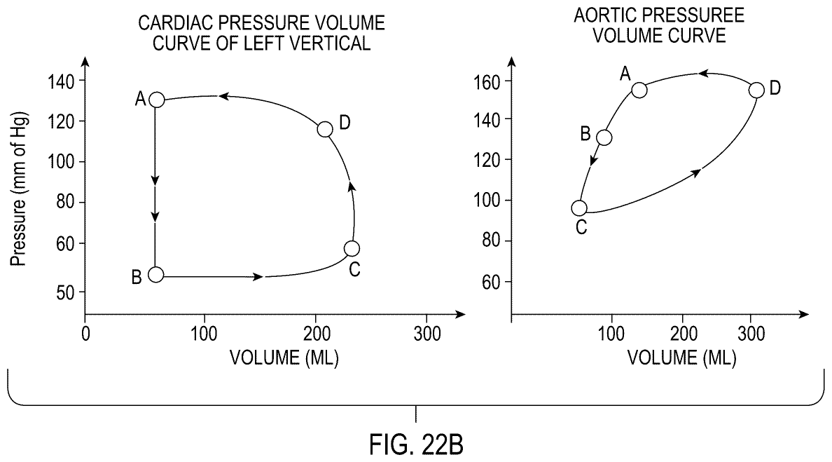

Mapping vessel or lumen dimensions and flow dynamics of tubes along with respect to liquids or solids that flow through the lumen or tube or valve: In the above example, blood viscosity and blood flow dynamics are being calculated. Upstream and downstream luminal dimensions and flow dynamics will be recorded during remodeling procedure. With reference to FIG. 22, in a lumen or tube such as a coronary artery a particular level of stenosis should be remodeled to achieve ideal luminal cross-section that is congruent with that what is upstream and downstream to the stenosis. It would be pointless to enlarge the stenotic region to a size larger than upstream to the stenotic region because it will only result in creating an upstream bottleneck. It could also result in creating the Bernoulli effect which would in turn alter tangential shear stress on endothelial wall and cause disproportionate endothelial growth response and accelerate restenosis. Mapping of flow dynamics in a stenotic vessel can distort the picture of what would be ideal flow dynamics after remodeling has restored the stenosis to normal lumen dimensions. We know that V1*A1=V2*A2, where V1 is velocity of Newtonian fluid in a tube with cross-sectional area A1 and V2 and A2 are for the narrower distal segment of the same tube (diagram on volume flow rate below). Pre-stenotic coronary lumen will have reduced velocities due to stenotic distal lesion (bottleneck) effect. Data acquired from aortic root fluid dynamics will be used by AI to aid in determining ideal velocities and transfer of blood volume (density, mass calculations) to forecast oxygen transfer to coronary artery after atherolysis aided opening of stenosis and how much atherolysis is needed to this ideal luminal cross-section. See FIG. 19,

The cap 180 of the catheter include myriad sensors, including IVUS, which are used to measure proximal and distal dimensions in a patient to reach conclusions about dimensions. Then, a computer, such as an AI driven computer system, performs calculations to assign the forecasted dimensions of the stenotic segment, and robotics guide a LASER to perform precise degree of remodeling of the aortic valve. The computer can map ranges of pressures exerted by flow dynamics on lumen or valve cusps, map maxima and minima for excursion of lumen or valve cusps during normal and extreme levels of flow. In a patient experiencing aortic stenosis it is not possible to directly visualize the normal excursion of the aortic valve cusps, therefore AI driven computer-generated forecasts of valve cusp dimensions are performed. Computer assisted graphics and AI will precisely create excursion of the valve cusps. Once one or more of the aortic cusps are remodeled, a tool can be used to perform excursions of the valve cusp to confirm adequate opening and adequate elasticity have been achieved to open and shut the cusp. This tool will measure thickness, elasticity, compute shear stress, excursion of valve under stress and under rest conditions, flexibility, and ability to close properly and seal the aortic inlet.

With reference to FIGS. 16, 22, and 23, the computer-generated forecasts can be based on various calculations, such as tensile force, elastic quotient, vector mapping of pressures, shear force, turbulence coefficient and viscosity of fluid & Bernoulli coefficient. Mapping of the ventricular structure and dimensions along with ventricular contractility data is useful to determine the end systolic volume (ESV) and end diastolic volume (EDV) of the left ventricle. Other data related to the rate of narrowing in the ventricular outflow tract will be used to forecast outflow tract velocities using algorithms to forecast velocities that will result from remodeling of the Aortic Valve. These will be tested by the equipment, sensors, etc. present in the cap 180 of the catheter. The computer-generated forecasts can include normalizing a stress rate and a strain rate at fluid-solid boundaries and tangential stress, calculating maxima, minima, and range of valve cusp thickness, calculating maxima, minima, and range of valve elasticity, distortion resistance, calculating maxima, minima, and range of valve range of motion, and calculating precise valve dimensions, shape, and size.

Referring to FIGS. 12 and 16, each cusp of the aortic valve has a generally triangular shape forming ⅓^{rd} the circumference of the aortic annulus in which the three valve cusps are seated forming an inverted Mercedes Benz sign at the center. Each cusp also has a curvilinear sweep from the periphery to the center in a way that the tip of each cusp curves caudal to cranial into the aortic root away from the ventricle. In cross-section, this forms a cardioid shape. Cardioid is a shape when a circle is drawn around a central circle using the same radius. It is observed that about half the length of each cusp towards the center of the aortic valve is curved as a cardioid while the remaining peripheral half is linear. The cardioid shape of the aortic valve cusp is an inverse curve of a parabola with the radius of the aortic valve annulus and about 25 degrees to 30 degrees of arc angle. AI and computer graphics will be used to build the frame of each cusp using this geometric principle.

As mentioned above, each cusp dimension will be constructed using computer graphic interface (CGI) and inputs from the cap 180 technology. An animation of all cusps, which includes pre-treatment animations of valve morphology and excursion and AI driven generation of proposed and predicted effects of LASER remodeling including mapping the thickness of all layers of the valve cusps and excursion of valves in response to various heart rates and blood flow velocity and pressure, will be created. CGI and repeat cycling decay simulation methods used in biomedical engineering to test longevity of product in accelerated short timeframes will be performed albeit in virtual space. Complex mathematical equations and programs will be used to carry out the necessary durability assessment of the valve cusps.

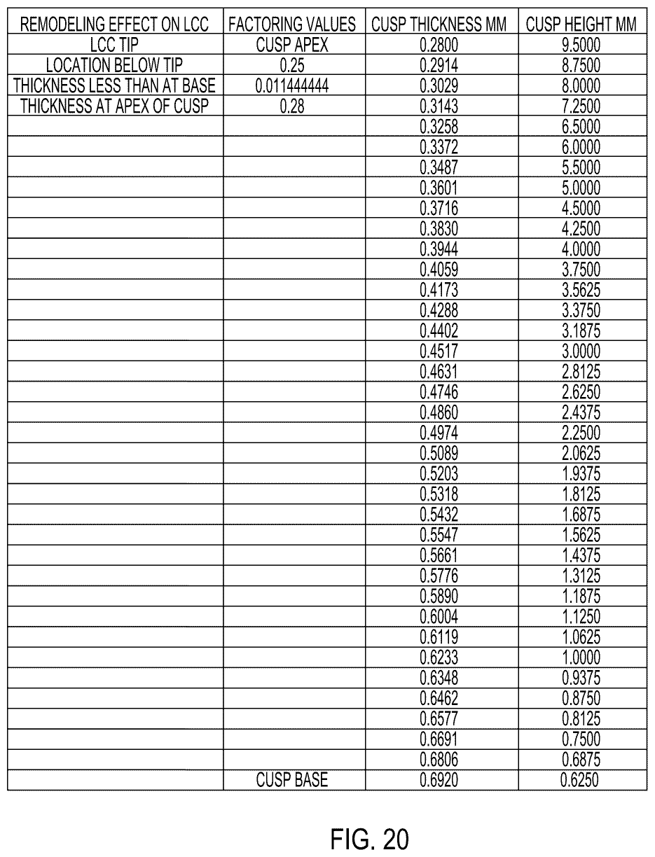

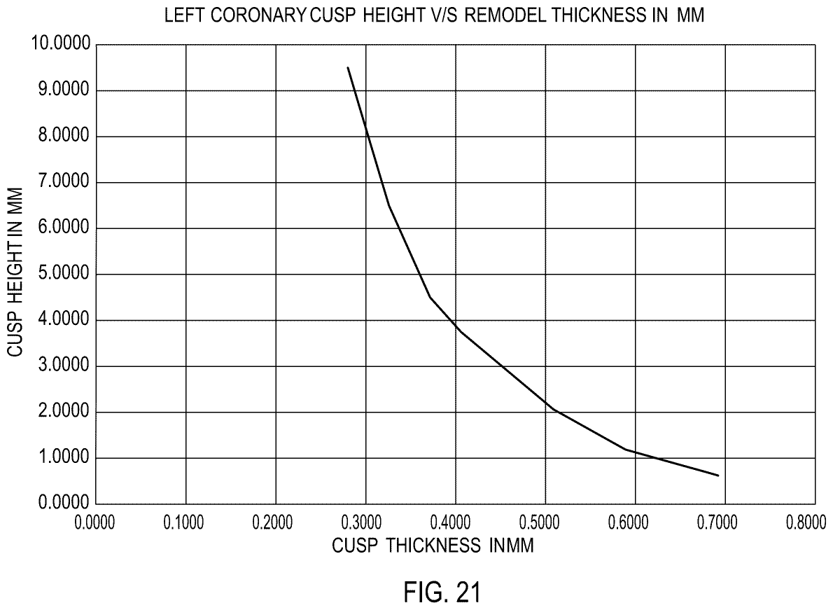

The method can also include computer-generated based calculations of ventricularis and aortic endothelial structure (thickness and/or smoothness) and morphology ideal for elasticity, solid-liquid surface friction/inertia, and prevention of fluidic restriction, stagnation, eddy currents, and degree of cusp sag that will create a tight seal during diastole to prevent valvular regurgitation. These are the final calculations performed as all the above inputs and functionalities are completed. Simulation of cusp sag when valves are closed in diastole. Impact of shear stress from column of blood in aorta will be confirmed to match the 3 mm sag that is expected from the proper thickness of the remodeled valve cusps using materials of appropriate types, appropriate amounts, density, and elastic properties. Calculations and resulting incremental increase in thickness of a cusp of the aortic valve, after remodeling, are demonstrated as shown in FIGS. 20 and 21. The thickness at the base of the cusp, where it is attached to the annulus, averages at 0.692 mm at a height of 0.625 mm from the lowest point on the annulus. Remodeling gradually decreases the thickness of the cusp from base to the apex of the cusp where the Node of Arantius is inserted and where the free tips of the three cusps come together during valve closure. The purpose of incremental thickening of the cusp from apex to base favors the impact of T (WSS) forces on valve cusp opening. The thicker base flexes almost none while the much thinner apex flexes the most in response to the ventricular outflow jet. The goal is to reach effective height and function of the cusps.

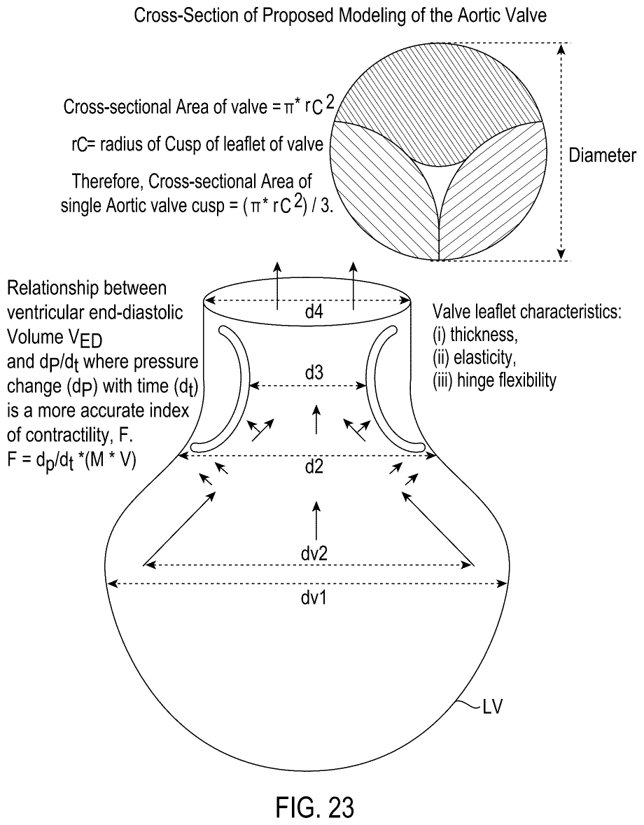

Referring now to FIG. 23, a top view and a cross-sectional view of the left ventricle is illustrated with dimensions noted for use in the mathematical equations set forth therein. Cross-sectional Area of single Aortic valve cusp=(π*rC2)/3, and the relationship between ventricular end-diastolic Volume V_{ED} and dP/dt where pressure change (dP) with time (dt) is a more accurate index of contractility, F.

$$F = dp / dt * ( M * V )$$

Factors affecting movement of cusps of leaflets during systole and diastole include:
1. Cusp edge rigidity
2. Cusp leaflet flexibility and ability to sag, close and open.
3. Blood velocity
4. Cross-sectional area of the aortic valve
5. Ventricular contractility
   wherein
Pₛ=systolic Pressure in left ventricle
P_{d}=Diastolic Pressure in left ventricle
T_{sys}=Systolic time Interval
M_{b}=Mass of blood (volume)
V_{b}=Blood flow velocity
d₄=Aortic diameter above the sinus of Valsalva
d₃=Diameter of aortic valve opening (systolic valve opening)
d₂=diameter at aortic valve annulus
dᵥ₁=Left Ventricular diameter in diastole
dᵥ₂=Left Ventricular diameter in systole
η=viscosity of blood.

An advantage of the system disclosed herein is the ability to create a complete separation of the ventricle from the aorta, secure the position of the first annular valve and second annular valves in the treatment segment of the catheter as described herein to provides unidirectional (ventricle to aorta) unimpeded flow of blood with each heartbeat while preventing backward blood flow from aorta into the ventricle (aortic regurgitation) during treatment of the cusp of the aortic valve. Another advantage is the ability to apply machine learning algorithms, artificial intelligence, and robotics integrated with data acquired from direct intra-luminal imaging, video, remodeling, and pre- and post-procedure direct visualization of aortic valve. This and other areas of the vascular system may be studied during insertion and withdrawal of the catheter without the use of contrast dye, which can cause damage to kidneys.

Although the invention is shown and described with respect to certain embodiments, modifications will occur to those skilled in the art upon reading and understanding the specification, and the present invention includes all such modifications.

What is claimed is:

1. A treatment segment of a catheter for heart valve treatment comprising:
a distal catheter body having a collapsible and expandable through conduit in surrounding relationship thereto and operatively connected to a collapsible and expandable valve cusp enclosure,
wherein the through conduit has a first end sealed by a first annular valve and a second end sealed by a second annular valve, and each of the first and second annular valves have an elastic body, a distal end connected to the valve cusp enclosure and a proximal end sealingly engaged to the distal catheter body in a deployed, closed position;
wherein the through conduit and the distal ends of each of the first and second annular valves are configured to expand with the valve cusp enclosure at a rate proportionate to a patient's blood pressure and a volume of blood displaced during expansion, and the elastic body and proximal ends of each of the first and second annular valves are configured to open and close in response to systolic and diastolic blood flow, respectively, thereby moving the proximal end of each in and out of sealing engagement with the distal catheter body.

2. The treatment segment of claim 1, wherein the elongate body of the through conduit is configured to retain blood between systolic opening and diastolic closing of the first and second annular valves.

3. The treatment segment of claim 1, wherein the first annular valve and the second annular valve are each frustoconically-shaped in a deployed, closed position.

4. The treatment segment of claim 1, wherein the valve cusp enclosure comprises:
one or more ventricular cusp and aortic cusp pairs, wherein each pair has attached ends at the through conduit, the attached ends being spaced apart and having a catheter working port exit therebetween, each pair defines a pocket therebetween, and each pair has free ends configured to engage a patient's inferior surface of the aortic valve and superior surface of the aortic valve, respectively.

5. The treatment segment of claim 4, wherein the catheter working port exit is a terminal end of a port that extends radially from the distal catheter body through the through conduit, wherein the port is expandable and collapsible between a deployed state and a transport state, respectively.

6. The treatment segment of claim 4, wherein the pocket is configured to receive a single one of a patient's valve cusp therein.

7. The treatment segment of claim 4, wherein the pocket is configured to receive two or more of a patient's valve cusps therein.

8. The treatment segment of claim 4, wherein the one or more ventricular cusp and aortic cusp pairs each comprise an inflatable balloon having a plurality of individually controlled inflation chambers.

9. The treatment segment of claim 8, wherein the aortic cusp of the one or more ventricular cusp and aortic cusp pairs is controllably inflatable to maintain blood flow to the right and left coronary arteries.

10. The treatment segment of claim 9, wherein the through conduit has a length configured to position the first annular valve superior relative to the right and left coronary arteries.

11. The treatment segment of claim 9, wherein the aortic cusp is controllably inflatable to increase blood flow to the left and right coronary arteries.

12. The treatment segment of claim 4, wherein the one or more ventricular cusp and aortic cusp pairs each comprise a plurality of telescoping, operatively expandable links housed inside a biomaterial.

13. The treatment segment of claim 12, wherein each of the plurality of telescoping, operatively expandable links terminate with a foot configured to engage the respective inferior or superior surface of the annulus of the aortic valve.

14. The treatment segment of claim 10, wherein each of the plurality of telescoping, operatively expandable links comprise one or more elbow joints defining an axis of rotation about which the more distal link is rotatable.

15. The treatment segment of claim 1, further comprising an electrocardiogram sensor; wherein the through conduit and the distal ends of each of the first and second annular valves are configured to expand controllably based on data from the electrocardiogram sensor.

16. A method of remodeling a cusp of a heart valve of a patient in need thereof, the method comprising:
introducing a catheter having a heart valve treatment segment according to claim 1 to a target heart valve;
deploying the collapsible and expandable valve cusp enclosure into an expanded state in which a cusp of a heart valve in need of remodeling is enclosed in an isolated pocket and simultaneously expanding the through conduit into a corresponding expanded state;
removing blood from the isolated pocket via the catheter to form a bloodless field surrounding the cusp of the heart valve;
remodeling the cusp of the heart valve;
collapsing the valve cusp enclosure; and
removing the catheter from the patient.

17. The method of claim 16, wherein introducing the catheter comprises feeding the catheter through a patient's artery based on robotics in a terminal cap guided by fiberoptic imaging or infrared or IVUS videography or EKG sensors that seek cardiac sinus node electric homing, or a combination thereof.

18. The method of claim 16, wherein deploying the valve cusp enclosure comprises inflating a plurality of balloon segments with a fluid.

19. The method of claim 18, wherein determining a lipid burden or calcium burden or fibrous scar tissue includes application of near-infrared spectroscopy plus intravascular ultrasound or a capacitive micromachines ultrasound transducer and fiberoptic cameras.

20. The method of claim 16, wherein the method further comprises advancing a tool through the catheter into the bloodless field and remodeling comprises one or more of:
i) removing fibrotic, calcific, and/or lipid laden material from a surface of the cusp using the tool;
ii) smoothing a surface of the cusp using the tool;
iii) preparing a surface of the cusp to accept a resurfacing material using the tool; and
iv) applying a resurfacing material to a surface of the cusp.

21. The method of claim 20, wherein removing comprises a laser treatment of the surface of the cusp.

22. The method of claim 20, wherein the resurfacing material comprises elastin and/or stem cells.

23. The method of claim 20, wherein the resurfacing material comprises a drug treatment.

24. The method of claim 23, wherein the drug treatment comprises collagen and/or carbon dots comprising stem cells, and the method further comprises activating the collagen by application of an activating wavelength of energy.
